(19) United States
(12) Patent Application Publication
(10) Pub. No.: US 2024/0344047 A1
(43) Pub. Date: Oct. 17, 2024

(54) MODIFIED RECOMBINANT COLLAGENASE, COMPOSITIONS COMPRISING THE SAME AND USES THEREOF IN DENTAL RELATED PROCEDURES

(71) Applicant: RAMOT AT TEL AVIV UNIVERSITY LTD., Tel Aviv (IL)

(72) Inventors: Maayan GAL, Tel Aviv (IL); Evgeny WEINBERG, Tel Aviv (IL); Inbal SHER, Tel Aviv (IL); Ran TOHAR, Tel Aviv (IL); Tamar ANSBACHER, Tel Aviv (IL)

(21) Appl. No.: 18/676,485

(22) Filed: May 28, 2024

Related U.S. Application Data
(63) Continuation of application No. PCT/IL2022/051304, filed on Dec. 11, 2022.
(60) Provisional application No. 63/289,263, filed on Dec. 14, 2021.

(51) Int. Cl. C12N 9/64; A61K 6/50; A61K 38/48; A61P 1/02

(52) U.S. Cl. CPC C12N 9/6489; A61K 6/50; A61K 38/4886; A61P 1/02; C12Y 304/24003

(57) ABSTRACT

Provided herein are modified forms of recombinant collagenase polypeptide having one or more amino acid substitutions and/or deletions compared to a wild type recombinant collagenase protein. Further provided are nucleic acid molecules encoding the modified recombinant collagenase polypeptide, compositions including the same and uses thereof in various dental related procedures.

### CROSS REFERENCE TO RELATED APPLICATIONS

This application is a Bypass Continuation of PCT Patent Application No. PCT/IL2022/051304 having International filing date of Dec. 11, 2022, which claims the benefit of priority of U.S. Provisional Patent Application No. 63/289,263, filed Dec. 14, 2021, the contents of which are all incorporated herein by reference in their entirety.

### REFERENCE TO AN ELECTRONIC SEQUENCE LISTING

The contents of the electronic sequence listing (015-PCT SEQ LISTING.xml; Size: 49,470 bytes; and Date of Creation: May 28, 2024) is herein incorporated by reference in its entirety.

### FIELD OF THE INVENTION

The present invention relates to modified forms of recombinant collagenase polypeptide having amino acid(s) substitution compared to a wild type collagenase protein. The invention further relates to compositions including the modified recombinant collagenase and uses thereof in various dental related procedures.

### BACKGROUND OF THE INVENTION

Collagen is the main structural protein in the extracellular matrix (ECM) in various connective tissues. Collagen is made of amino acids forming a triple helix of elongated fibril, also termed a collagen helix. Several types of Collagen are known, including, Fibrillar (Type I, II, III, V, XI) and Non-fibrillar. Collagen can be enzymatically degraded by enzymes, such as Collagenases, which break the peptide bonds in collagen.

Dental teeth extraction or exodontia is a fundamental procedure performed in dental surgery. Ultimately, exodontia should allow controlled and safe removal of teeth, leading to a complete healing with no post-operative prosthetic problem. Basic principles of tooth extraction haven't changed dramatically for several decades. Typically, disruption of periodontal ligament (PDL) fibers from the bundle bone of the socket and primary tooth luxation are achieved employing dental elevator, followed by further socket expansion and delivery of the tooth with dental forceps. Therefore, tooth extraction is an invasive procedure, often characterized by a difficult to avoid collateral damage to surrounding tissues. Furthermore, in order to successfully remove the tooth, reflection of a mucoperiosteal flap and removal of alveolar bone is often required to visualize and gain access to the teeth roots, thus enhancing the trauma caused by extraction. Different intra- and postoperative complications are associated with tooth extractions such as root fracture, bleeding and hemorrhage, displaced teeth, bone fracture, soft tissue injury, damage to adjacent tooth, infection, alveolar osteitis and paresthesia. Moreover, tooth extraction is associated with osteonecrosis of the jaw in a patient with a history of antiresorptive (e.g., bisphosphonates) or antiangiogenic agents. Furthermore, rehabilitation with dental implants may become more complex whether hard and soft tissues are not preserved during the tooth extraction. This leads to an increasing demand in lowering damage to soft and hard tissues around the tooth being extracted. Atraumatic or minimally invasive tooth extraction may reduce intra- and postoperative complications and maintain bone and soft tissues for subsequent implant placement and restoration.

Current techniques of minimally invasive tooth extraction either implement mechanical disruption of PDL fibers, such as periotomes and Piezosurgery or limit the extraction forces to the vertical dimension thus minimizing socket expansion. However, vertical extraction systems are suitable mainly for the single-rooted teeth, entirely depend on the retention of the screw in the root canal and may cause root fracture thus complicating the extraction procedure. Another tool designed for atraumatic tooth extraction is physics forceps, which decreases the crown, root, and buccal bone plate fractures in comparison to the conventional forceps, relying principally on mechanical advantage.

Collagen is considered the main structural component of PDL. Hence, a promising approach for minimally invasive tooth extraction is the degradation of the collagen bundles which establish continuity across the ligament and anchor the tooth to the bundle bone. A natural machinery for cleaving collagen is the family of collagenases. These are essential enzymatic elements of the matrix metalloproteinase (MMP) family of proteins (Nagase, H., et. al., Cardiovascular Research, 2006. 69(3): p. 562-573). Their main function is degradation of collagen, designated to maintain the balance of the connective tissue components. Among the various types, bacterial collagenases are known as efficient enzymes capable of degrading triple helix collagen and breaking it down to short peptide fragments. Indeed, given their efficient activity, application of collagenase of Clostridium histolyticum is approved for the treatment of Dupuytren's and Peyronie's diseases. In the context of dental medicine, it was previously shown that collagenase reduces the area of collagen fibers and strength of the PDL (Kawada, J. and K. Komatsu, Japanese Journal of Oral Biology, 2000. 42(3): p. 193-205; Komatsu, K., et. al., J Biomech, 2007. 40(12): p. 2700-6). Zinger A. et. al., (ACS Nano 2018, 12, 1482-1490) discloses proteolytic nanoparticles (of collagenase) can replace a surgical blade by controllably remodeling the oral connective tissue.

Ducka p. et. al. (Applied Microbiology and Biotechnology volume 83, 1055-1065 (2009) is directed to universal strategy for high-yield production of soluble and functional clostridial collagenases in E. coli, and discloses recombinant collagenase polypeptides.

U.S. Pat. No. 10,016,492 is directed to methods of extracting teeth involving contacting, prior to extraction, the tissue surrounding a tooth to be extracted with a composition providing an agent capable of destroying the periodontal ligament surrounding the tooth, such as, collagenase.

Nevertheless, there is a need in the art for modified forms of recombinant collagenase, that exhibit improved properties compared to wild type collagenase, and which can be used in various dental related conditions in a robust, safe, efficient and cost-effective manner.

### SUMMARY OF THE INVENTION

According to some embodiments, there are provided advantageous recombinant collagenase polypeptides, a truncated recombinant collagenase, as well as novel modified recombinant collagenase polypeptides, which include one or more point mutations and/or truncations, compared to a wild-type (non-modified) collagenase. According to some embodiments, the non-naturally occurring, modified recombinant collagenases disclosed herein are advantageous, as they are stable, easy to produce, and exhibit a desired biological activity, as further detailed herein. Further provided are nucleic acid molecules encoding the modified recombinant collagenase polypeptides, methods for their preparations, compositions comprising the same and uses thereof in various dental related conditions for tooth and orthodontic procedures, including, for example tooth extraction.

According to some embodiments, the advantageous modified/non-naturally occurring/genetically modified/mutated recombinant collagenase polypeptides include at least one point mutation and/or truncation, compared to a WT, unmodified collagenase.

In some embodiments, the modified recombinant collagenases may include one or more truncations and/or one or more amino acid substitutions, in particular surface exposed amino acids. In some embodiments, the one or more amino acid substitutions may include the substitution of hydrophobic (or at least less hydrophilic) amino acids to more hydrophilic or charged amino acids.

In some embodiments, as exemplified hereinbelow, the modified recombinant collagenases exhibit increased solubility and/or thermal resistance, as compared to a WT collagenase protein.

In some exemplary embodiments, a modified recombinant collagenase may include one or more of the following amino acid substitutions (compared to a WT peptide): N149Q, M183D, N203Y, N287Y, F295Y, A334D, A458P, S353H, D536P, T635N, Q669D, G670N, G672T, S701N, A709E, E710H and D737K. In some embodiments, hydrogen bonds (H-bonds) form. For example—A709E with Y693 from an adjacent α helix. Furthermore, a hydrogen bond network may also form, for example H710-N419-E709. Moreover, N203Y substitution may form pi-pi stacking with Tyr150 from a neighboring loop. Additionally, the inclusions of prolines may backbone stability. Salt bridges and pi cation interactions as well as hydrophobic interactions may also stabilize the protein.

In some embodiments, the modified collagenase may include at least one, at least two, at least three, at least four, at least five, at least six, at least seven, at least eight, at least nine, at least ten, at least eleven, at least twelve, at least thirteen, at least fourteen, at least fifteen amino acid substitutions. Each possibility is a separate embodiment.

According to some embodiments, the modified collagenase peptides may be used in procedures of various dental related conditions, such as, but not limited to: tooth extractions (in particular, minimally invasive tooth extraction), orthodontics, removal of implants, hypertrophic lesions in the oral mucosa, prevention of post-orthodontic treatment relapse, non-surgical crown lengthening procedures, decontamination of the tooth or implant surface, and the like, or any combination thereof.

In some exemplary embodiments, the modified collagenase peptides may successfully be used in enzymatically assisted exodontia by being delivered to the periodontal ligament. As exemplified herein, the local delivery/administration of the modified collagenase peptides resulted in an efficient method to significantly reduce the required force for tooth extraction. In some embodiments, a significant reduction of at least 20%, at least 30%, at least 40%, at least 50% in the applied force was observed.

According to some embodiments, there is provided herein an innovative approach to analytical monitoring of extraction force showing a clear relation in reduction in the force required for tooth extraction following enzymatic-based disruption of PDL fibers with modified recombinant collagenase.

According to some embodiments, as exemplified herein, utilizing the methods and compositions disclosed herein, atraumatic tooth extraction may be obtained, while reducing intra- and post-operative complications and facilitating subsequent implant placement.

According to some embodiments, minimally invasive extraction has important implications for reducing intra- and post-operative complications, particularly in medically compromised patients, such as those with disorders of hemostasis, and/or treated with antiresorptive or antiangiogenic agents. In some embodiments, atraumatic extraction may also be important for successful prosthetic rehabilitation with dental implants, since it may facilitate preservation of adjacent hard and soft tissues. This may diminish referrals of patients to secondary care settings such as an oral surgery unit, and also decrease patients' stress and anxiety levels.

According to some embodiments, enzymatic degradation of collagen may further have clinical relevance for the treatment of hypertrophic lesions in the oral mucosa, such as irritation fibroma and gingival fibromatosis, prevention of post-orthodontic treatment relapse (by replacing the use of a surgical scalpel for the procedure known as circumferential supracrestal fiberotomy), non-surgical crown lengthening procedures, decontamination of the tooth or implant surface.

In some embodiments, a wild type (non-modified) collagenase polypeptide may have an amino acid sequence as denoted SEQ ID NO: 10.

In some embodiments, a modified recombinant collagenase polypeptide may have an amino acid sequence as denoted by any one of SEQ ID NO: 3, SEQ ID NO: 5, SEQ ID NO: 7, SEQ ID NO: 13, SEQ ID NO: 15 or SEQ ID NO: 17. Each possibility is a separate embodiment. In some embodiments, a recombinant collagenase polypeptide may have a truncation of an N-terminal region thereof has an amino acid sequence as denoted by SEQ ID NO: 1.

According to some embodiments, as further exemplified herein, the advantageous modified recombinant collagenase may be thermally stable as compared to a WT collagenase.

In some embodiments, the modified recombinant collagenases disclosed herein, for example, a modified recombinant collagenase may have an amino acid sequence as denoted by SEQ ID NO: 3, may exhibit improved activity in dental related procedures, as compared to a WT collagenase and/or to a recombinant collagenase may have a truncation of the N-terminus thereof. In some embodiments, the modified recombinant collagenase may exhibit one or more improved properties as compared to a WT collagenase, the properties may include: pharmacologic effects, pharmacokinetic, stability, half-life, efficiency, stability, side effects, and the like, or any combination thereof.

According to some embodiments, provided herein are methods and compositions for treatment of dental related conditions, the methods include administration of a modified recombinant collagenase (may have a truncation and/or one or more point mutations), or a composition comprising the same to a subject in need thereof. In some embodiments, the dental related condition may be selected from extraction, orthodontic conditions and removal of implants.

According to some embodiments, the modified recombinant collagenase polypeptide may have an amino acid sequence as denoted by SEQ ID NO: 3. According to some embodiments, the modified recombinant collagenase polypeptide may have an amino acid sequence as denoted by SEQ ID NO: 5. According to some embodiments, the modified recombinant collagenase polypeptide may have an amino acid sequence as denoted by SEQ ID NO: 7. In some embodiments, a modified recombinant collagenase polypeptide may have an amino acid sequence as denoted by SEQ ID NO: 1.

According to some embodiments, there is provided a composition comprising a modified recombinant collagenase polypeptide (may have a truncation/deletion and/or one or more point mutations) disclosed herein.

According to some embodiments, the modified recombinant collagenase polypeptide disclosed herein, or the composition comprising the same may be used for treating a tooth-related condition in a subject in need thereof.

According to some embodiments, there is provided a modified recombinant collagenase polypeptide, said modified recombinant collagenase polypeptide may include one or more amino acid replacements relative to the corresponding wild type collagenase amino acid sequence and/or one or more truncations.

According to some embodiments, there is provided a modified recombinant collagenase polypeptide, said modified recombinant collagenase polypeptide may include one or more amino acid replacements relative to the corresponding wild type collagenase amino acid sequence. In some embodiments, the modified recombinant collagenase may further include an N-terminal truncation compared to the corresponding WT collagenase. In some embodiments, the N-terminal truncation may include a truncation of at least 15, at least 50, at least 100 amino acids relative to the corresponding wild type collagenase amino acid sequence.

According to some embodiments, the modified recombinant collagenase polypeptide may include an amino acid sequence as denoted by any one of SEQ ID NO: 3, 5, 7, 13, 15 or 17. In some embodiments, the modified recombinant collagenase polypeptide may include an amino acid sequence as denoted by SEQ ID NO: 3 or 13.

According to some embodiments, the modified recombinant collagenase polypeptide may further include a Tag sequence at the N-terminus and/or the C-terminus thereof. In some embodiments, the Tag sequence may be used for marking/identification and/or purification of the modified recombinant collagenase. In some embodiments, the tag sequence may be selected from His tag (i.e., including a stretch of Histidine amino acids, for example, 8 Histidine amino acids), FLAG-tag, Myc-tag, GST-tag, GB1-tag and the like. The tag sequences may be placed in-frame at the N-terminal of the modified proteins and/or on the N-terminal of the modified protein. In some embodiments, a linker may be placed between the tag sequence and the collagenase polypeptide sequence.

According to some embodiments, the thermal stability of the modified recombinant collagenase polypeptide may be higher than the thermal stability of the corresponding wild type polypeptide.

According to some embodiments, there is provided a composition which may include a modified recombinant collagenase polypeptide as disclosed herein.

According to some embodiments, the modified recombinant collagenase polypeptide or the composition comprising the same may be used in dental related procedures in a subject in need thereof.

According to some embodiments, the dental related procedures may be selected from tooth extraction, orthodontic procedure and implant removal.

According to some embodiments, the modified recombinant collagenase polypeptide or the composition including the same, may be administered prior to, during or after the dental related procedure.

According to some embodiments, the modified recombinant collagenase polypeptide or the composition may be administered locally. In some embodiments, the administration may be by local injection. According to some embodiments, the administration may reduce extraction force during tooth extraction.

According to some embodiments, the required force for extraction after administration of modified collagenase or the composition including the same, may be reduced compared to administration of a WT collagenase, a recombinant collagenase (having an N-terminus truncation), or to administration of a carrier.

According to some embodiments, the modified recombinant collagenase polypeptide or the composition may be administered locally to the PDL region prior to the tooth extraction.

According to some embodiments, there is provided a method of treating a dental related condition in a subject in need thereof, the method may include administering to the subject in need thereof an effective amount of the modified recombinant collagenase polypeptide or the composition including the same. According to some embodiments, the administration may be local administration.

According to some embodiments, there is provided a method of extracting a tooth in a subject in need thereof, the method may include administering to the subject in need thereof an effective amount of the modified recombinant collagenase polypeptide or the composition including the same; and applying a force after a period of time, to extract the tooth, wherein the administration may be performed by injection to tissue in the vicinity of the tooth. In some embodiments, the tissue may be around the root. In some embodiments, the tissue may be PDL. In some embodiments, the period of time may be between 1-120 minutes, 60 minutes-4 hours, 2 hours-24 hours, 2.5 hours-36 hours, 1 minute-48 hours, and the like. In some embodiments, the dose of administration may be in the range of about 0.1 μg/μl to about 200 μg/μl, or any subranges thereof.

According to some embodiments, there is provided a nucleic acid molecule encoding the modified recombinant collagenase disclosed herein. In some embodiments, the nucleic acid molecule may have a nucleotide sequence as denoted by any one of SEQ ID NO: 4, SEQ ID NO: 6, SEQ ID NO: 8, SEQ ID NO: 14, SEQ ID NO: 16, or SEQ ID NO: 18. In some embodiments, the nucleic acid molecule may have a nucleotide sequence as denoted by SEQ ID NO: 4.

According to some embodiments, there is provided a vector including the nucleic acid molecule encoding the modified recombinant collagenase as disclosed herein. In some embodiments, the vector may be an expression vector, further including one or more regulatory sequences.

According to some embodiments, there is provided a host cell harboring the nucleic acid molecule encoding the modified recombinant collagenase as disclosed herein.

According to some embodiments, there are provided host cells transformed or transfected with the vector disclosed herein.

According to some embodiments, there is provided a host cell harboring the modified recombinant collagenase polypeptides disclosed herein.

According to some embodiments, there is provided a method of treating a dental related condition in a subject in need thereof, the method may include administering to the subject in need thereof a therapeutically effective amount of the modified recombinant collagenase polypeptide disclosed herein, or a composition including the same.

According to some embodiments, there is provided a method of treating a dental related condition in a subject in need thereof, the method may include administering to the subject in need thereof a therapeutically effective amount of a modified recombinant collagenase polypeptide, or a composition including the same, wherein the modified recombinant collagenase polypeptide may include a truncation of an N-terminus thereof and optionally one or more amino acid substitutions or deletions, as compared to a corresponding WT collagenase.

According to some embodiments, there is provided a method of producing the modified recombinant collagenase polypeptide, the method may include the steps of: (i) culturing the host cells under conditions such that the polypeptide comprising the modified recombinant collagenase is expressed; and (ii) optionally recovering the modified recombinant collagenase from the host cells or from the culture medium.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-E Show pictograms of experimental setting of porcine jaw for tooth extraction. FIG. 1A—Left side of a pig lower jaw after removal of soft tissues. Premolar teeth are marked as PM1 and PM2; FIG. 1B After root splitting; Teeth are marked as T1 (mesial root of PM1), T2 (distal root of PM1), T3 (mesial root of PM2) and T4 (distal root of PM2); FIG. 1C—Injection of recombinant collagenase (truncated or modified (including additional one or more mutations) using a computerized device; FIG. 1D—pictograms of the jaw fixed in the self-designed devise installed over tensile strength testing machine; FIG. 1E pictogram of the tooth root after extraction;

FIG. 2A Graphs showing the evaluation of force vs. displacement during tooth extraction, with or without treatment with recombinant collagenase. For each root (T1-T4), the force vs. displacement was recorded in real time, during extraction following the injection of a recombinant Collagenase (ColG, having an N-terminus truncation) or PBS (control). Four exemplified extraction curves are shown;

FIG. 2B shows a pictogram of the anatomical structure of roots T1, T2, T3 and T4 after extraction;

FIGS. 3A-C show graphs of average extraction force of ColG, and PBS treated tooth roots. FIG. 3A—Type specific average extraction force of the maximum recorded force for T1-4 tooth roots across the different jaws. Blue and red colors represent ColG, and PBS treated tooth roots, respectively. FIG. 3B Nonspecific average extraction force of all tooth roots that were treated with ColG (red) vs PBS (blue); FIG. 3C a graphic presentation of mean and dispersion of extraction forces of ColG and PBS treated tooth roots;

FIGS. 4A-B show graphs of extraction force of distinct tooth roots. FIG. 4A—Each panel represents a different type of tooth root T1-T4. Vertical dots show the extraction force for tooth roots in the same jaw that were treated with ColG (red/dark gray) or PBS (blue/light gray). FIG. 4B—graphs showing mean force difference between PBS and ColG treated roots;

FIG. 5 shows lines graphs of thermal stability of modified recombinant recombinase (Des1) compared to a truncated recombinant collagenase (ColG). A heat inactivation assay was performed by preincubating the purified recombinant proteins, at temperatures ranging between 35 and 90° C. for 1h. Residual activity was then measured by monitoring collagenase activity. The midpoint of temperature inactivation, the temperature at which 50% of the activity was retained (T50), was determined by fitting a two-state model using GraphPad;

FIG. 6—graphs showing force required to extract the different roots following the administration (injection) of PBS, recombinant collagenase G (ColG) or a modified recombinant collagenase (Des1).

FIGS. 7A-C—graphs showing exemplary cellular viability of CHO cells and hGFs treated with ColG, wherein (FIG. 7A) CHO and (FIG. 7B) primary human gingival fibroblast viability. Cells were treated with variable concentrations of ColG, and cellular viability was evaluated. Results are shown relative to PBS-treated cells. (FIG. 7C) Cells that were treated with PBS or 5 mg/mL ColG were re-plated with a fresh medium, and cellular viability was evaluated again. Results are relative to cells originating from PBS-treated cells.

FIGS. 8A-C—exemplary photographs relating to cellular toxicity, wherein hGFs treated with (FIG. 8A) 5 mg/mL ColG, (FIG. 8B) PBS or (FIG. 8C) 70% ethanol. Green cells are viable and red cells are dead. The indicated scale bar shows 100 m length.

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As referred to herein, the terms “polynucleotide molecules”, “oligonucleotide”, “polynucleotide”, “nucleic acid” and “nucleotide” sequences may interchangeably be used. The terms are directed to polymers of deoxyribonucleotides (DNA), ribonucleotides (RNA), and modified forms thereof in the form of a separate fragment or as a component of a larger construct, linear or branched, single stranded (ss), double stranded (ds), triple stranded (ts), or hybrids thereof. The polynucleotides may be, for example, or polynucleotide sequences of DNA or RNA. The DNA or RNA molecules may be, for example, but are not limited to: complementary DNA (cDNA), genomic DNA, synthesized DNA, recombinant DNA, or a hybrid thereof or an RNA molecule such as, for example, mRNA. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent inter nucleoside linkages, as well as oligonucleotides having non-naturally occurring portions, which function similarly to respective naturally occurring portions. As used herein, nucleotides (A, G, C or T) and nucleotide sequences are marked in lowercase letters (a, g, c or t)

The terms “polypeptide,” “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the polypeptide may contain modification, such as but be not limited only to, glycosylation, phosphorylation or disulfide bond shape. Also provided are conservative amino acid variants of the peptides and protein molecules disclosed herein. Variants according to the invention also may be made that conserve the overall molecular structure of the encoded proteins or peptides. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved. As used herein, amino acids and peptide sequences are marked using conventional amino acid nomenclature (single letter or 3-letters code). For example, amino acid “Serine” may be marked as “Ser” or “S” and amino acid “Cysteine” may be marked as “Cys” or “C”.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A or a) forms a base pair with thymine (T or t) and guanine (G or g) with cytosine (C or c). In RNA, thymine is replaced by uracil (U or u). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

The term “construct”, as used herein refers to an artificially assembled or isolated nucleic acid molecule which may be comprises of one or more nucleic acid sequences, wherein the nucleic acid sequences may be coding sequences (that is, sequence which encodes for an end product), regulatory sequences, non-coding sequences, or any combination thereof. The term construct includes, for example, vectors, plasmids but should not be seen as being limited thereto. The term “regulatory sequence” in some embodiments, refers to DNA sequences, which are necessary to affect the expression of coding sequences to which they are operably linked (connected/ligated). The nature of the regulatory sequences differs depending on the host cells. For example, in prokaryotes, regulatory/control sequences may include promoter, ribosomal binding site, and/or terminators. For example, in eukaryotes regulatory/control sequences may include promoters (for example, constitutive of inducible), terminators enhancers, transactivators and/or transcription factors. A regulatory sequence which is “operably linked” to a coding sequence is ligated in such a way that expression of the coding sequence is achieved under suitable conditions. In some embodiments, a “Construct” or a “DNA construct” refer to an artificially assembled or isolated nucleic acid molecule which comprises a coding region of interest and optionally additional regulatory or non-coding sequences.

As used herein, the term “vector” refers to any recombinant polynucleotide construct (such as a DNA construct) that may be used for the purpose of transformation, i.e. the introduction of heterologous DNA into a host cell. One exemplary type of vector is a “plasmid” which refers to a circular double stranded DNA loop into which additional DNA segments can be ligated. Another exemplary type of vector is a viral vector, wherein additional DNA segments can be ligated into the viral genome. Certain vectors are capable of autonomous replication in a host cell into which they are introduced. The term “Expression vector” refers to vectors that have the ability to incorporate and express heterologous nucleic acid fragments (such as DNA) in a foreign cell. In other words, an expression vector comprises nucleic acid sequences/fragments (such as DNA, mRNA), capable of being transcribed or expressed in a target cell. Many viral, prokaryotic and eukaryotic expression vectors are known and/or commercially available. Selection of appropriate expression vectors is within the knowledge of those having skill in the art. The expression vectors can include one or more regulatory sequences. The expression vectors can encode for a recombinant protein. The expression vector can be used for transient transfection or to generate permanent/stable line.

As used herein, a “primer” defines an oligonucleotide which is capable of annealing to (hybridizing with) a target nucleotide sequence, thereby creating a double stranded region which can serve as an initiation point for DNA synthesis under suitable conditions.

As used herein, the term “transformation” refers to the introduction of foreign DNA into cells. The terms “transformants” or “transformed cells” include the primary transformed cell and cultures derived from that cell regardless to the number of transfers. All progeny may not be precisely identical in DNA content, due to deliberate or inadvertent mutations. Mutant progeny that have the same functionality as screened for in the originally transformed cell are included in the definition of transformants.

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, injection, and the like, or combinations thereof. The transfection of the cell may be performed on any type of cell, of any origin, such as, for example, human cells, animal cells, plant cells, and the like. The cells may be isolated cells, tissue cultured cells, cell lines, cells present within an organism body, and the like.

According to some embodiments, a subject in need thereof (i.e., a subject that needs to undergo a dental relate procedure) may be a human subject, any other mammal or non-mammal animal, such as, for example, farm animals, pets, and the like.

The term “wild type collagenase”, “WT collagenase”, “and “un-modified collagenase” may interchangeably be used. The terms refer to a form of collagenase having an identical amino acid sequence as that of a corresponding WT collagenase. In some embodiments, the WT-recombinant collagenase is from a non-mammalian origin. In some embodiments, the WT-recombinant collagenase is from a mammalian origin. In some embodiments, the WT-recombinant collagenase is of clostridium origin. In some embodiments, the WT-recombinant collagenase is of human origin. In some embodiments, the WT-recombinant collagenase has an amino acid sequence as denoted by SEQ ID NO: 10. The polynucleotide sequence as set forth in SEQ ID NO: 9 corresponds to the cDNA encoding the WT recombinant collagenase as set forth in SEQ ID NO: 10, corresponding to Accession No. Q9X721.

As used herein the terms “recombinant collagenase”, “truncated recombinant collagenase”, and “ColG” may interchangeably be used. The terms relate to a truncated form of the corresponding WT Collagenase, having a truncation of at least 20 amino acids of the N-terminus. In some embodiments, the recombinant collagenase is from a non-mammalian origin. In some embodiments, the recombinant collagenase is from a mammalian origin. In some embodiments, the WT-recombinant collagenase is of clostridium origin. In some embodiments, the recombinant collagenase includes an amino acid sequence as denoted by SEQ ID NO. 1. In some embodiments, the recombinant collagenase includes an artificial N-terminus region including a His-tag and protease cleavage site. In some embodiments, the artificial N-terminal region has an amino acid sequence as denoted by SEQ ID NO: 11.

As used herein the terms “modified recombinant collagenase”, “mutated recombinant collagenase”, “non-naturally occurring recombinant collagenase”, and “Des” may interchangeably be used. The terms relate to a mutated/modified form of the corresponding wild-type (WT) or natural form of the recombinant collagenase, or of a corresponding recombinant collagenase (i.e., ColG, having an N-terminus truncation). In some embodiments, the modified recombinant collagenase is of clostridium. In some embodiments, the modified recombinant collagenase is of non-mammalian origin. In some embodiments, the modified recombinant collagenase is of mammalian origin. In some embodiments, the modified recombinant collagenase differs from the corresponding wild type collagenase by at least one mutation selected from amino acid substitution(s), and/or deletions(s). In some embodiments, the modified recombinant collagenase, includes an amino acid sequence as denoted by SEQ ID NO. 3 (also referred to herein as Des1). In some embodiments, the modified recombinant includes an amino acid sequence as denoted by SEQ ID NO. 5 (also referred to herein as Des4). In some embodiments, the modified recombinant collagenase, includes an amino acid sequence as denoted by SEQ ID NO. 7 (also referred to herein as Des6). In some embodiments, a modified recombinant collagenase of an origin other than Clostridium may include a corresponding point mutation and/or deletion in the respective WT collagenase, which are equivalent or homologous to the mutations introduced in the Clostridium WT collagenase. In some embodiments, the modified recombinant Collagenase may further include an artificial N-terminus region including a tag (such as a His Tag) and protease cleavage site. In some embodiments, the artificial N-terminal region has an amino acid sequence as denoted by SEQ ID NO: 11. In some embodiments, the modified recombinant collagenase, includes an amino acid sequence as denoted by SEQ ID NO. 13. In some embodiments, the modified recombinant collagenase, includes an amino acid sequence as denoted by SEQ ID NO. 15. In some embodiments, the modified recombinant collagenase, includes an amino acid sequence as denoted by SEQ ID NO. 17.

As used herein, the term “isolated” means either: 1) separated from at least some of the components with which it is usually associated in nature with respect of the Wild-Type collagenase; 2) prepared or purified by a process that involves the hand of man; 3) not occurring in nature.

In some embodiments, there is further provided a nucleic acid molecule encoding a polypeptide comprising an amino acid sequence of a modified recombinant collagenase. In some embodiments, there is provided a nucleic acid molecule having a nucleotide sequence as denoted by SEQ ID NO: 4, encoding a polypeptide having an amino acid sequence of a modified recombinant collagenase having an amino acid sequence as denoted by SEQ ID NO: 3. In some embodiments, there is provided a nucleic acid molecule having a nucleotide sequence as denoted by SEQ ID NO: 6, encoding a polypeptide having an amino acid sequence of a modified recombinant collagenase having an amino acid sequence as denoted by SEQ ID NO: 5. In some embodiments, there is provided a nucleic acid molecule having a nucleotide sequence as denoted by SEQ ID NO: 8, encoding a polypeptide having an amino acid sequence of a modified recombinant collagenase having an amino acid sequence as denoted by SEQ ID NO: 7. In some embodiments, the nucleic acid molecule encoding a polypeptide having an amino acid sequence of a modified recombinant collagenase, further includes a nucleotide sequence encoding for the artificial N-terminal region, the artificial N-terminal region has a nucleotide sequence as denoted by SEQ ID NO: 12. In some embodiments, there is provided a nucleic acid molecule having a nucleotide sequence as denoted by SEQ ID NO: 14, encoding a polypeptide having an amino acid sequence of a modified recombinant collagenase having an amino acid sequence as denoted by SEQ ID NO: 13. In some embodiments, there is provided a nucleic acid molecule having a nucleotide sequence as denoted by SEQ ID NO: 16, encoding a polypeptide having an amino acid sequence of a modified recombinant collagenase having an amino acid sequence as denoted by SEQ ID NO: 15. In some embodiments, there is provided a nucleic acid molecule having a nucleotide sequence as denoted by SEQ ID NO: 18, encoding a polypeptide having an amino acid sequence of a modified recombinant collagenase having an amino acid sequence as denoted by SEQ ID NO: 17.

In some exemplary embodiments, the nucleic acid molecule encoding for the modified recombinant collagenase disclosed herein is preferably at least 50% homologous/identical to the nucleic acid sequence as shown in SEQ ID NO: 2. It is understood that such nucleic acid sequences can also include orthologous/homologous/identical (and thus related) sequences. More preferably, the nucleic acid sequence encoding the provided modified recombinant collagenase is at least 52%, 53%, 55%, 60%, 65%, 70%, 75%, 80%, 85%, 90%, 91%, 92%, 93%, 94%, 95%, 96%, 97%, 98%, or 99% homologous/identical to the nucleic acid sequence as shown in SEQ ID NO: 2, wherein the higher values of sequence identity are preferred.

In some exemplary embodiments, the nucleic acid molecule encoding for the modified recombinant collagenase disclosed herein is preferably at least 60% homologous/identical to the nucleic acid sequence as shown in SEQ ID NO: 4 or 14. It is understood that such nucleic acid sequences can also include orthologous/homologous/identical (and thus related) sequences. More preferably, the nucleic acid sequence encoding the provided modified recombinant collagenase is at least, 65%, 70%, 75%, 80%, 85%, 90%, 91%, 92%, 93%, 94%, 95%, 96%, 97%, 98%, or 99% homologous/identical to the nucleic acid sequence as shown in SEQ ID NO: 4 or 14, wherein the higher values of sequence identity are preferred. In some exemplary embodiments, the nucleic acid molecule encoding for the modified recombinant collagenase disclosed herein is preferably at least 60% homologous/identical to the nucleic acid sequence as shown in SEQ ID NO: 6 or 16. It is understood that such nucleic acid sequences can also include orthologous/homologous/identical (and thus related) sequences. More preferably, the nucleic acid sequence encoding the provided modified recombinant collagenase is at least, 65%, 70%, 75%, 80%, 85%, 90%, 91%, 92%, 93%, 94%, 95%, 96%, 97%, 98%, or 99% homologous/identical to the nucleic acid sequence as shown in SEQ ID NO: 6 or 16, wherein the higher values of sequence identity are preferred. In some exemplary embodiments, the nucleic acid molecule encoding for the modified recombinant collagenase disclosed herein is preferably at least 60% homologous/identical to the nucleic acid sequence as shown in SEQ ID NO: 8 or 18. It is understood that such nucleic acid sequences can also include orthologous/homologous/identical (and thus related) sequences. More preferably, the nucleic acid sequence encoding the provided modified recombinant collagenase is at least, 65%, 70%, 75%, 80%, 85%, 90%, 91%, 92%, 93%, 94%, 95%, 96%, 97%, 98%, or 99% homologous/identical to the nucleic acid sequence as shown in SEQ ID NO: 8 or 18, wherein the higher values of sequence identity are preferred.

According to some embodiments, the recombinant or the modified recombinant collagenase may further include a protein tag. As used herein, the term “protein tag” refers to a peptide sequence bound to the N-terminus or C-terminus of the protein. According to some embodiments, the protein tag may include a glycoprotein. According to some embodiments, the protein tag may be used for separation, purification and/or identification/tracking of the tagged protein. Non-limiting examples of protein tags include: Myc-Tag, Human influenza hemagglutinin (HA), Flag-Tag, His-Tag, Glutathione-S-Transferase (GST) and a combination thereof. Each possibility represents a separate embodiment of the present invention. In some embodiments, the protein tag is His-tag.

According to some embodiments, the modified recombinant collagenase may include a protein tag upon production, which may be consequently cleaved and/or removed from the produced recombinant collagenase prior to incorporation into a composition or prior to being introduced to cells/administered. Cleavage and/or removal of a tag may be performed by any method known in the art, such as, but not limited to, enzymatic and/or chemical cleaving. In some embodiments, the cleavage may be facilitated by a cleavage site included the amino acid sequence. In some embodiments, the recombinant and/or the modified recombinant collagenase include at the N-terminus a tag sequence and a cleavage site. In some embodiments, an amino acid sequence as denoted by SEQ ID NO: 11 includes a His tag and a protease cleavage site.

According to some embodiments, the modified recombinant collagenase as disclosed herein may be produced by recombinant methods from genetically-modified host cells. Any host cell known in the art for the production of recombinant proteins may be used for the present invention. According to some embodiments, the host cell is a prokaryotic cell. Representative, non-limiting examples of appropriate prokaryotic hosts include bacterial cells, such as cells of Escherichia coli and Bacillus subtilis. According to other embodiments, the host cell may be a eukaryotic cell. According to some exemplary embodiments, the host cell may be a fungal cell, such as yeast.

According to some exemplary embodiments, a coding region of interest is a coding region encoding WT-Recombinant collagenase. According to some exemplary embodiments, a coding region of interest is a coding region encoding modified recombinant collagenase. According to some exemplary embodiments, a coding region of interest is a coding region encoding for a modified recombinant collagenase as set forth in SEQ ID NOs: 4, 6 or 8.

In some embodiments, the modified recombinant collagenase may be synthesized by expressing a polynucleotide molecule encoding the modified recombinant collagenase in a host cell, for example, a microorganism cell transformed with the nucleic acid molecule.

In some embodiments, DNA sequences encoding wild type polypeptides, may be isolated from any cell producing them, using various methods well known in the art. For example, a DNA encoding the wild-type polypeptide may be amplified from genomic DNA by polymerase chain reaction (PCR) using specific primers, constructed on the basis of the nucleotide sequence of the known wild type sequence. The genomic DNA may be extracted from the cell prior to the amplification using various methods known in the art.

According to some embodiments, a polynucleotide encoding ta modified recombinant collagenase polypeptide may be cloned into any vector known in the art.

According to some embodiments, upon isolation and/or cloning of the polynucleotide encoding the wild type polypeptide, desired mutation(s) may be introduced by modification at one or more base pairs, using methods known in the art, such as for example, site-specific mutagenesis, cassette mutagenesis, recursive ensemble mutagenesis and gene site saturation mutagenesis. Methods are also well known for introducing multiple mutations into a polynucleotide. For example, introduction of two and/or three mutations may be performed using commercially available kits, such as the QuickChange site-directed mutagenesis kit (Stratagene). In some embodiments, as exemplified herein, point mutations may be introduced into the sequence encoding for the Recombinant collagenase (represented by SEQ ID NO: 2), and/or a WT collagenase (represented by SEQ ID. NO: 9).

According to some embodiments, the polynucleotide thus produced may then be subjected to further manipulations, including one or more of purification, annealing, ligation, amplification, digestion by restriction endonucleases and cloning into appropriate vectors. The polynucleotide may be ligated either initially into a cloning vector, or directly into an expression vector that is appropriate for its expression in a particular host cell type.

In some embodiments, in case of a fusion protein, or a protein fused with a protein tag, different polynucleotides may be ligated to form one polynucleotide. In some embodiments, the polynucleotide encoding the recombinant or modified recombinant collagenase polypeptide, may be incorporated into a wide variety of expression vectors, which may be transformed into in a wide variety of host cells.

According to some embodiments, introduction of a polynucleotide into the host cell may be effected by well-known methods, such as chemical transformation (e.g. calcium chloride treatment), electroporation, conjugation, transduction, calcium phosphate transfection, DEAE-dextran mediated transfection, transvection, microinjection, cationic lipid-mediated transfection, scrape loading, ballistic introduction and infection. Representative, non-limiting examples of appropriate hosts may include bacterial cells, such as cells of E. coli and Bacillus subtilis.

In some embodiments, the polypeptides may be expressed in any vector suitable for expression. The appropriate vector is determined according to the selected host cell. Vectors for expressing proteins in E. coli, for example, include, but are not limited to, pET, pK233, pT7 and/or lambda pSKF. Other expression vector systems are based on betagalactosidase (pEX); maltose binding protein (pMAL); and glutathione S-transferase (pGST).

According to some embodiments, selection of a host cell transformed with the desired vector may be accomplished using standard selection protocols involving growth in a selection medium which is toxic to non-transformed cells. For example, in the case of E. coli, it may be grown in a medium containing an antibiotic selection agent; cells transformed with the expression vector which further provide an antibiotic resistance gene, may grow in the selection medium. In some embodiments, upon transformation of a suitable host cell, and propagation under conditions appropriate for protein expression, the polypeptide may be identified in cell extracts of the transformed cells. Transformed hosts expressing the polypeptide may be identified by analyzing the proteins expressed by the host, for example, using SDS-PAGE and comparing the gel to an SDS-PAGE gel obtained from the host which was transformed with the same vector but not containing a nucleic acid sequence encoding the desired polypeptide.

According to some embodiments, the desired polypeptides which have been identified in cell extracts may be isolated and purified by conventional methods, including ammonium sulfate or ethanol precipitation, acid extraction, salt fractionation, ion exchange chromatography, hydrophobic interaction chromatography, gel permeation chromatography, affinity chromatography, and the like, and combinations thereof. The polypeptides of the invention may be produced as fusion proteins, attached to an affinity purification protein tag, such as a His-tag, in order to facilitate their rapid purification.

According to some embodiments, there is provided a process for the production of a modified recombinant collagenase polypeptide the process may include culturing/raising suitable host cells under conditions allowing the expression of the modified recombinant collagenase polypeptide and optionally recovering/isolating the produced polypeptide from the cell culture.

According to some embodiments, there is provided a nucleic acid encoding for the modified recombinant collagenase polypeptide. In some embodiments, there is provided a DNA construct/vector (such as, an expression vector) harboring or comprising a nucleic acid encoding for the modified recombinant collagenase polypeptide (optionally in addition to one or more regulatory sequences, non-coding sequences, and the like).

In some embodiments, various suitable vectors are known to those skilled in art, and the choice of which depends on the function desired. Such vectors may include, for example, plasmids, cosmids, viruses, bacteriophages and other vectors. In some embodiments, the polynucleotides and/or vectors harboring the same may be reconstituted into vehicles, such as, for example, liposomes for delivery to target cells. Any cloning vector and/or expression vector known in the art may be used, depending on the purpose, the host cell, and the like. Such vectors may be used for in-vitro and/or in-vivo introduction/expression.

According to some embodiments, there is provided a host cell harboring or expressing the modified recombinant collagenase. In some embodiments, the host cell may be transformed/transfected with a vector or with a nucleic acid encoding for the modified recombinant collagenase. In some embodiments, there is provided a host cell harboring and/or comprising the nucleic acid molecule encoding for a modified recombinant collagenase. In some embodiments, the presence of at least one vector or at least one nucleic acid molecule in the host may mediate the expression of the modified recombinant collagenase in the cell. In some embodiments, the nucleic acid molecule or vector comprising the same, may either integrate into the genome of the host cell, or it may be maintained extrachromosomally. In some embodiments, the host cell may be any prokaryotic or eukaryotic cell.

According to some embodiments the nucleic acid molecules may be used alone or as part of a vector to express the modified recombinant collagenase polypeptide of the invention in cells, for purification and/or for therapy (i.e., use in dental related procedures).

According to some embodiments, there is provided a composition which includes the recombinant or modified recombinant collagenase polypeptide, the nucleic acid encoding therefor, or vectors harboring the nucleic acids. Each possibility is a separate embodiment. In some embodiments, the composition may include one or more suitable excipients, according to the purpose, type and/or use of the composition. In some embodiments, excipient is a pharmaceutical excipient which may include or a pharmaceutical carrier, vehicle, buffer and/or diluent. In some exemplary embodiments, the composition may include carriers (such as, liposomal carriers) harboring or encapsulating the modified recombinant collagenase peptide or nucleic acid encoding the same.

According to some embodiments, the recombinant collagenase and/or the modified-recombinant collagenase (polypeptide or nucleic acid encoding the same) may be used successfully in various dental related conditions procedures.

According to some embodiments, any suitable route of administration to a subject may be used for a nucleic acid, polypeptide and/or the composition of the present invention. In some embodiments, the administration may be local. According to another embodiment, administration of the composition may be via an injection. For administration via injection, the composition may be formulated in an aqueous solution, for example in a physiologically compatible buffer, or in any suitable carrier, such as, liposomal carriers. Formulations for injection may be presented in unit dosage forms, for example, in ampoules, or in multi-dose containers with, optionally, an added preservative.

According to some embodiments, aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles. According to some embodiments, compositions for injection may contain formulatory agents such as suspending, stabilizing, and/or dispersing agents.

According to some embodiments, the recombinant and/or modified recombinant collagenase polypeptide, the nucleic acid encoding the same, and/or the composition comprising the polypeptide, when used in dental related procedures may be used in combination with other suitable agents. The components of such combinations may be administered sequentially or simultaneously/concomitantly in separate and/or combined formulations by any suitable administration route.

According to some embodiments, there is provided a method of treating a dental related condition, the method may include administration (for example, local administration) to a subject in need thereof a therapeutically effective amount of a modified recombinant collagenase. In some embodiments, the modified recombinant collagenase may be administered as a polypeptide as is, or in a suitable composition.

According to some embodiments, there are provided kits comprising the recombinant and/or the modified recombinant collagenase peptide and/or the nucleic acid molecule encoding the same and/or the composition as disclosed herein. Such a kit may be used, for example, in the treatment of various dental related conditions, such as, for example, extraction, orthodontic procedures, tooth replacement, implants removal, and the like.

According to some embodiments, the polypeptide or a composition including the same may be administered prior to, during or after the dental procedures. For example, the polypeptide or the composition may be administered 1-240 minutes, or any sub-range therein, prior to the procedure (for example, extraction). For example, the polypeptide or the composition may be administered 5-120 minutes prior to the procedure (for example, extraction). For example, the polypeptide or the composition may be administered 15-90 minutes prior to the procedure (for example, extraction). For example, the polypeptide or the composition may be administered 1 minutes-48 hours prior to the procedure (for example, extraction).

According to some embodiments, a prototype of a fixed porcine jaw in a self-designed anchoring device may be used in a tensile strength testing machine. The tensile strength testing machine may gradually increases the extraction force while monitoring the force versus displacement of the extracted tooth root. This setup may enable studying the effect of enzymatic disruption of the periodontal ligament via local administration of the recombinant collagenase peptides (WT and modified) as disclosed herein.

According to some embodiments, as exemplified herein, the biologically rationalized application of recombinant or modified recombinant collagenase may efficiently reduce the force required for tooth extraction. A significant reduction of up to 50% in the applied force may be observed.

According to some embodiments, as exemplified herein, there is provided a recombinant collagenase enzyme-based treatment for minimally invasive exodontia. According to some embodiments, as exemplified herein, there is provided a recombinant modified collagenase enzyme-based treatment for minimally invasive exodontia is feasible.

According to some embodiments, as exemplified herein, enzymatic disruption (utilizing recombinant collagenase and/or modified recombinant) of the periodontal ligament may reduce the force required for tooth extraction.

According to some embodiments, as exemplified herein, it was shown that disruption of periodontal ligament fibers with recombinant collagenase and/or modified recombinant collagenase may substantially reduce the force required for tooth extraction.

According to some embodiments, as exemplified herein, utilizing the methods and compositions disclosed herein, atraumatic tooth extraction may be obtained, while reducing intra- and post-operative complications and facilitating subsequent implant placement.

According to some embodiments, as exemplified herein, a large degree of variability in the forces may be required to extract the same type of root across different jaws.

In the description and claims of the application, the words “include” and “have”, and forms thereof, are not limited to members in a list with which the words may be associated. As used herein, the term comprising includes the term consisting of.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Expression and Purification of Recombinant Collagenase G (WT or Modified Forms)

Expression of WT collagenase G (ColG) was assessed as described (Ducka p. et. al. (Applied Microbiology and Biotechnology volume 83, 1055-1065 (2009); Tohar R. et. al., Int. J. Mol. Sci. 2021, 22, 8552).

Briefly, the plasmid containing ColG gene derived from clostridium histolyticum, comprising amino acids 119-880 with a TEV cleavable N-terminus His×6 tag, was transformed into E. coli BL21. Cells were grown to OD(600 nm)=0.8 and protein expression was induced by supplementing 1 mM IPTG for 16 h at 25° C. Following lysis of bacterial cells, the supernatant was passed onto a nickel column; and following extensive wash, the protein was eluted with 300 mM imidazole. Following buffer exchange to phosphate buffered saline (PBS, Biological Industries, Beit HaEmek, Israel), ColG was kept frozen with the addition of 50% glycerol.

Modified forms of recombinant collagenase were similarly prepared.

### Experimental Set-Up and Jaw Preparation

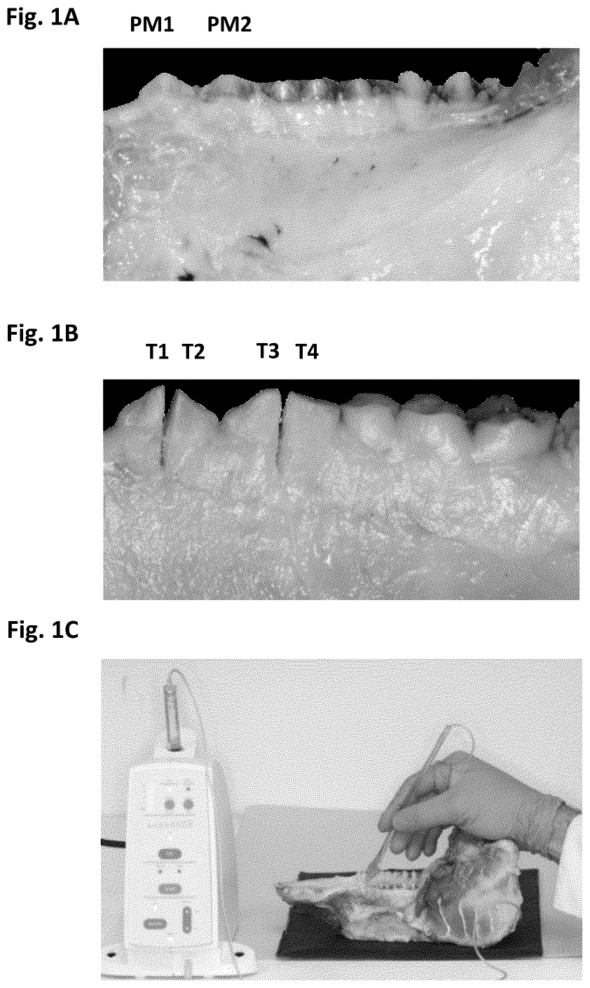

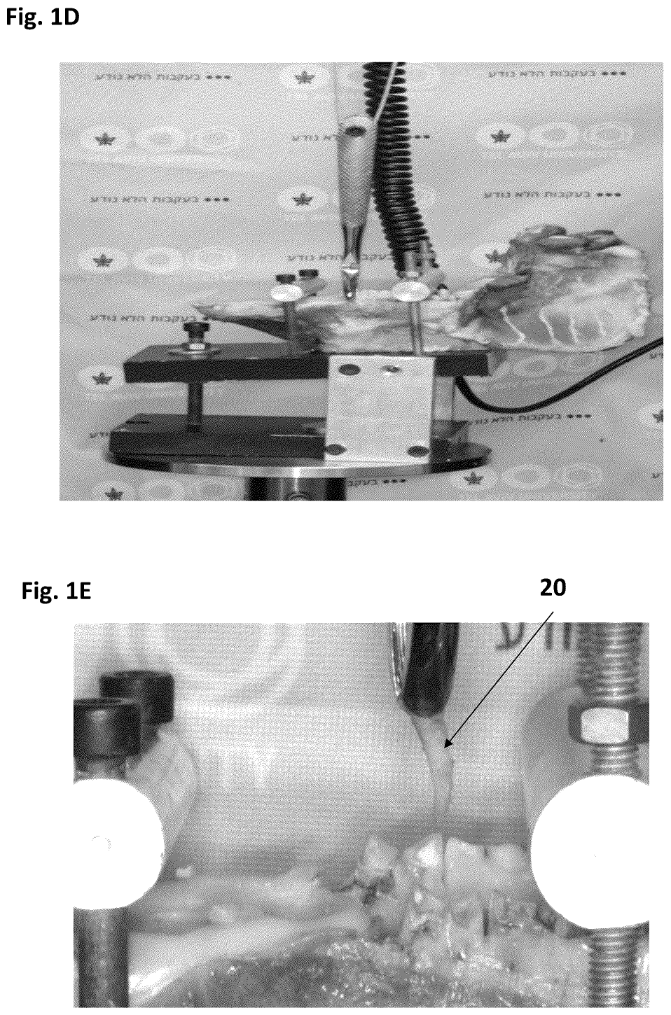

Twelve whole mandibles of six-month-old (90-100 kg) domestic swine were obtained proximate to slaughter. Only healthy samples with unimpaired teeth, gingival tissues, and alveolar mucosa were selected. A total of twelve jaws were selected for the experiments on the premolar teeth. Roots of the two premolar teeth (PM1 and PM2, FIG. 1A) to be extracted during the experiment were split using dental handpiece and high-speed diamond bur (Strauss & Co, Ra'anana, Israel). These were marked as T1 (mesial root of PM1), T2 (distal root of PM1), T3 (mesial root of PM2), and T4 (distal root of PM2) in FIG. 1B. Subsequently, mandibles were randomly divided by a split-mouth design into two sides, recombinant or modified recombinant collagenase, or PBS (a vehicle of that served as a negative control). One mesial and one distal root of PM1 (T1 and T2) broke prematurely and were excluded from the data, together with their contralateral counterparts. Data were obtained for the following final numbers of root contralateral pairs: eleven T1, eleven T2, twelve T3, and twelve T4.

### Injection of Collagenase to the PDL

Recombinant collagenase or PBS were injected with the Wand Single Tooth Anesthesia System (Milestone Scientific, New Jersey, USA) (FIG. 1C). Standard cartridges containing the local anesthetic solution for dental injection were accurately emptied of their content and filled with either recombinant or modified recombinant collagenase at a concentration of 4 μg/μl or PBS solution, followed by application to each treated root. Injection was performed using a 30G 2.54 cm length needle that was inserted into the PDL space and advanced apically until stopped by resistance of the alveolar bone proper. The injection was repeated at four sites around each root, on the buccal, lingual, mesial, and distal aspects. A total of 0.3 ml of 4 ug/ul recombinant or modified recombinant collagenase or PBS solution was injected.

### Tooth Extraction Anchoring Device

A device anchoring to the lower jaw of the tensile strength testing machine (Instron Series 4500; Instron Corp., Canton, Mass., USA) was specifically self-designed, with two parallel height-adjustable cylinders for securing the mandible anterior and posterior parts to the tooth to be extracted (shown in the pictogram of FIG. 1D). Moreover, the device 10 was designed to enable fixation of the porcine mandible under varying inclinations, so that the longitudinal axes of the tooth root could be adjusted perpendicular to the ground surface, thus limiting the extraction forces to the vertical dimension. Straight upper incisor and canine extraction forceps (Hu Friedy, Chicago, IL, USA), with drilled holes in the distal edges of the handles and a pin connecting them, were attached to the upper jaw of the tensile strength testing machine via a pull rope and locked firmly over a clinical crown of the root to be extracted with a screw.

### Measurement of Tooth Extraction Force

When the tooth crown or roots broke, the data from both the broken and the contralateral tooth roots were not included. Subsequently, the extraction forces of a total of eleven T1, T2 and twelve T3, T4 mandibles were recorded. The tensile load was applied to the tooth root by the tensile strength testing machine using a load cell of 10 kN and cross-head speed of 10 mm/min. Tensile force and displacement during the experiment were acquired via the designated software (Instron Series IX; Instron Corp., Canton, Mass., USA) at a rate of 10 Hz.

### Data Analysis

The data was analyzed, and the graphs presented in the figures were plotted with GraphPad prism 7 (RRID:SCR_002798). P-values were calculated using a paired T-test with Gaussian distribution.

### Example 1—Tooth Root Preparation and Injection of Recombinant Collagenase

To this aim, a standardized model for direct measurement of the force applied during tooth extraction was based on an ex-situ porcine jaw, which is recognized as a validated model for a range of dental applications. FIGS. 1A-D summarize the entire process, from jaw preparation to extraction. The jaws were carefully debrided from the soft tissues including gingiva adjacent to the teeth, thus exposing the tooth roots up to the alveolar crest (FIG. 1A). FIG. 1B shows the first and second premolars after splitting in the area of furcation into mesial and distal halves, marked as T1, T2, T3, and T4. The results show that owing to the root properties, splitting the premolars an essential step to reduce the required extraction force to a human comparable strength. Otherwise, the magnitude of the force would have been too high, which would have frequently lead to the breakage of the tooth crown or the instrumental setup. In the current setup, in which the application of extraction force was vertically applied along the longitudinal axes of the tooth root, it was enabled to pull the mesial and distal roots of porcine PM1 and PM2 intact from their sockets. WT recombinant collagenase (ColG) or PBS (control) was injected to the periodontal ligament (PDL) surrounding each tooth using the Wand system, as depicted in FIG. 1C. This enabled controllable and precise injection of the recombinant enzyme to the PDL in a clinically accepted approach. After an incubation period of about 16 hours at room temperature, which was intended to enable maximum enzymatic activity, the extraction forces were measured as detailed in Example 2.

### Example 2—Qualitative and Quantitative Analysis of Extraction Forces

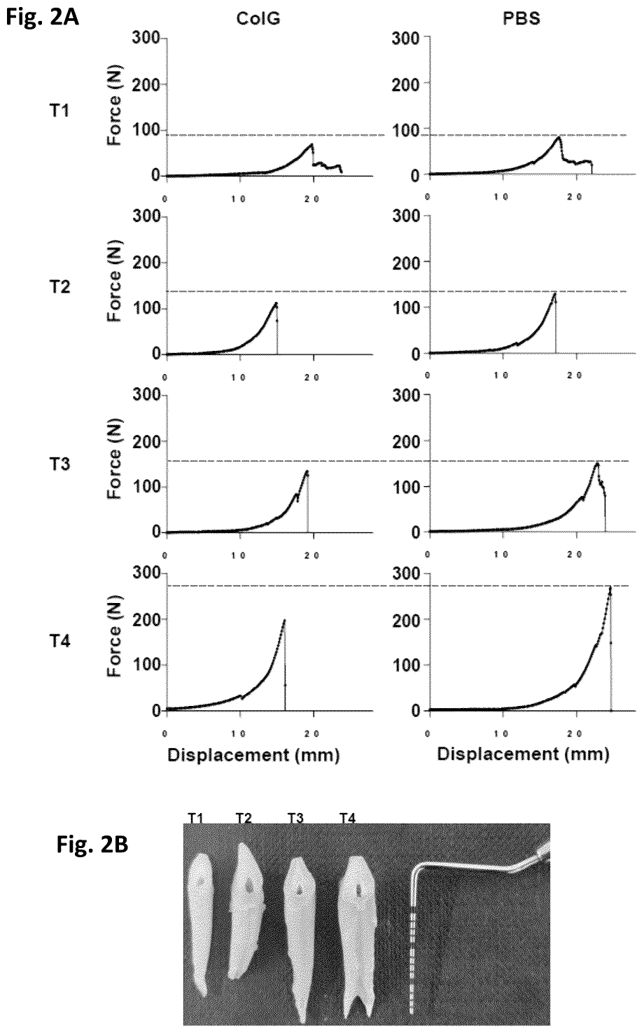

The extraction forces of the four roots were measured by real-time recording of the applied force on the examined root and its displacement. Each jaw was placed in the anchoring device, fixing its position while enabling direct vertical access such that each treated root was separately extracted (FIGS. 1D-1E (exemplary extracted root 20 is shown in FIG. 1E)). The results are presented in FIG. 2A, which shows four representative curves of force versus distance for each root that was extracted after the application of ColG or PBS.

A different lag distance in which the pulling cable reached a complete stretch was observed in each of the curves; this yielded apparently varied distance for the different roots. Several extraction patterns were observed. From the representative curves, it is evident that T1 had the lowest extraction force. In addition, T1 showed a unique pattern; specifically, following the maximal peak after which the root was extracted, the force did not immediately drop to zero but rather to a secondary baseline. This suggests that following root extraction from the PDL, soft tissues were still connected the root to the alveolar ridge, probably due to incomplete removal. Roots T2 and T3 demonstrated an overall similar extraction pattern, with a comparable maximal pulling force. T4 required application of the highest force to extract it. In addition to the pattern shown in FIG. 2A, an additional pattern was observed in several T4s that followed the first peak. This included an additional lower peak that was observed due to proximity-related friction of the T4 crown to the first molar tooth. It is enlightening to view the results in the context of the physical properties of the roots. FIG. 2B shows the anatomical diversities between representative roots. Root T1 is the narrowest, but longer than T2. The low surface area attached to the PDL resulted in relatively low extraction force. Although root T2 was the shortest, it was wider than T1 and similar to T3, but had two fused canals, thus showing comparable force to the longer T3. Root T4 was the widest, with two distinguished separated canals. Accordingly, T4 required application of the highest extraction force.

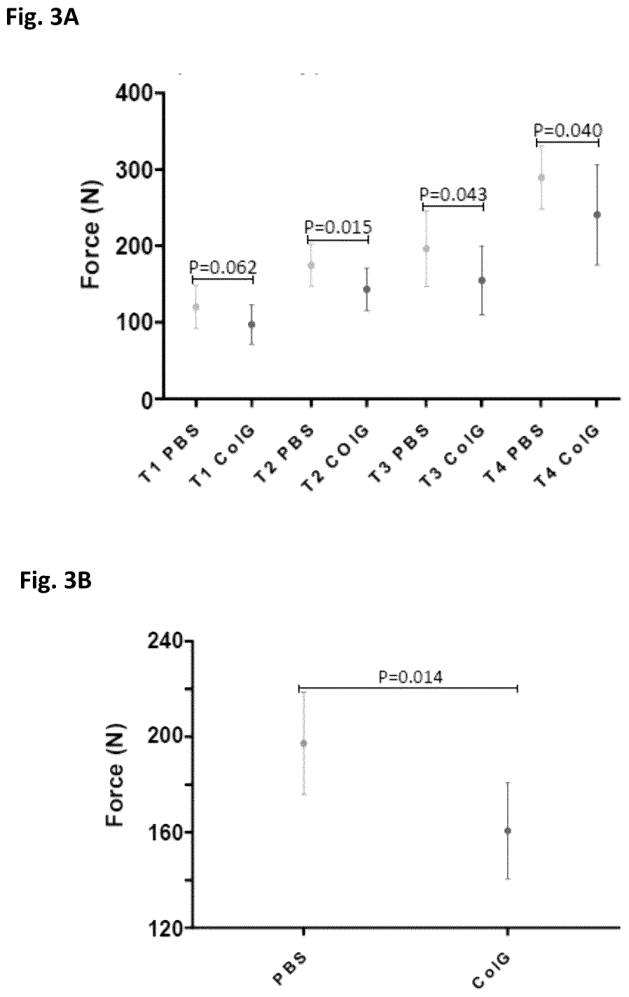

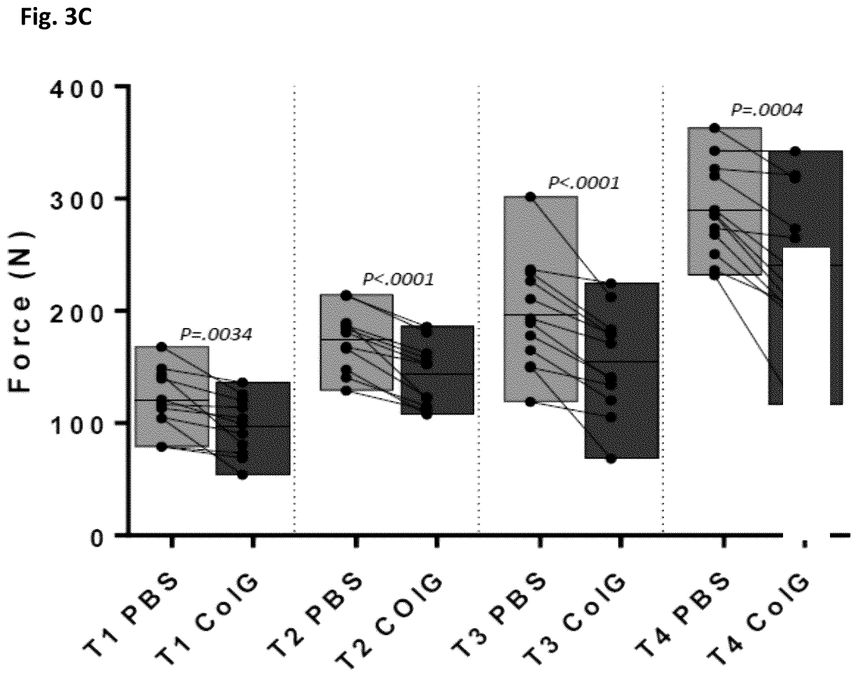

To evaluate the effect of injected ColG on the extraction force, the mean extraction force was calculated for each root type. FIG. 3A shows the mean force (marked by a horizontal black line), as well as the dispersion of the maximal force applied to extract roots T1-4 in all jaws following treatment with PBS (blue) or ColG (red). Lines connect the ColG and PBS treated roots in the same jaw. A clear reduction in the mean force was observed for each root type following treatment with ColG relative to PBS. Despite the clear reduction in force, the dispersion of maximum force was relatively high. This was due to the considerably large degree of variability in the physiological and morphological characteristics of PDL, thus corroborating observations in human jaws (Dietrich et al., 2020; Muska et al., 2013).

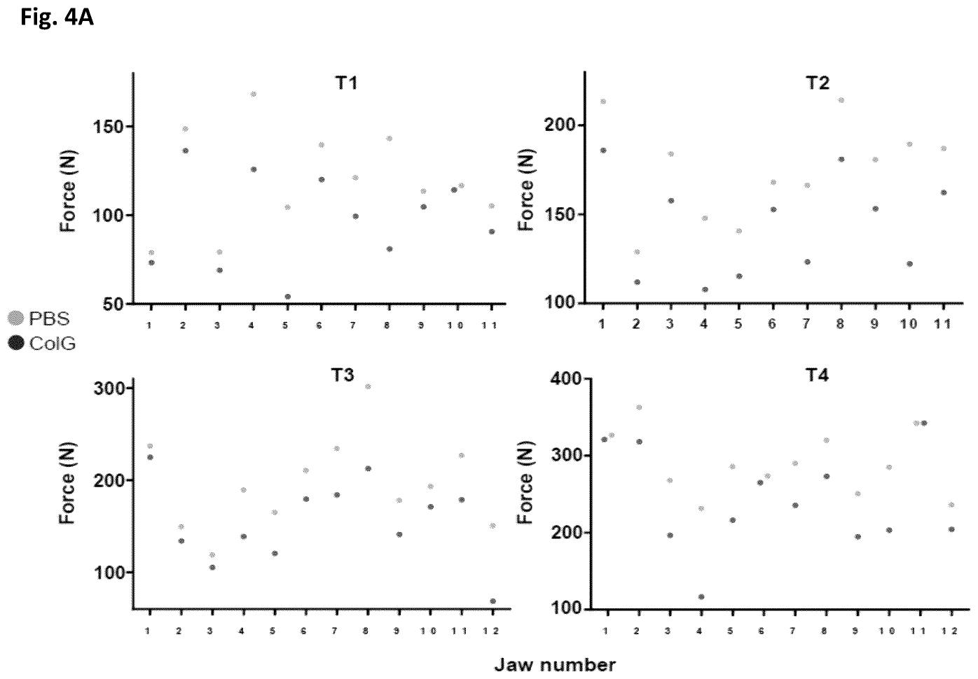

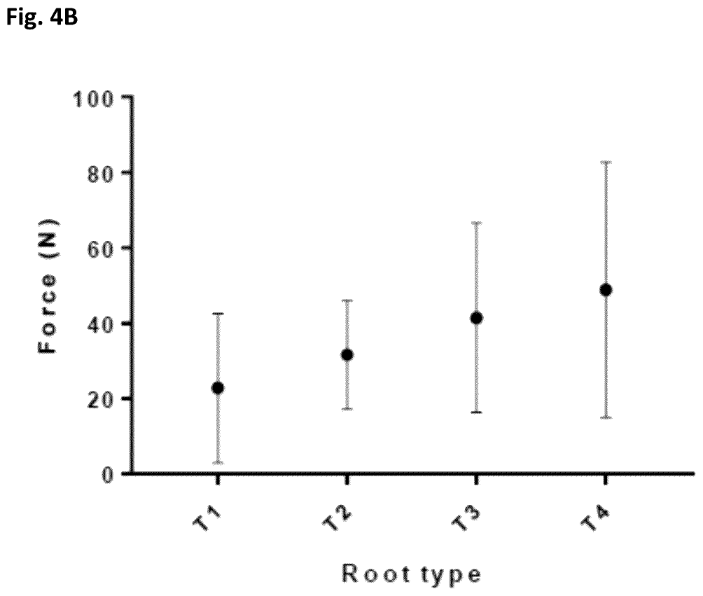

The force of treated vs. non-treated contralateral tooth roots in the same jaw was tested. FIG. 4A shows the maximal extraction force for T1 (top left), T2 (top right), T3 (bottom left) and T4 (bottom right), across the different jaws. Overall reduction in the applied force was observed in almost all the roots. In some roots, 50% reduction in the required force was recorded (e.g., T4 in jaw #4, T1 in jaw #8). However, in a minority of incidences, only negligible differences were detected (e.g., T1 in jaw #10, T4 in jaw #1, T4 in jaw #11). Of the 44 extracted roots, 20 showed >20% reduction in the applied force following treatment with ColG. Only five tooth roots showed <5% difference (FIG. 4B). Roots with only slight differences in force between the ColG and PBS treatments were typically part of the same jaw (e.g., jaw #1). FIG. 4B shows the mean difference in the applied extraction force between paired roots, treated with ColG versus PBS, for all jaws. Although the error was relatively high, a clear and clinically significant reduction in force was observed following the enzymatic application of ColG.

### Example 3: Construction of a Modified Recombinant Collagenase Protein

A modified recombinant collagenase, which contained point mutations in the corresponding WT sequence was generated. The modified recombinant collagenases were derived from wild type collagenase (or from a recombinant collagenase), using standard genetic engineering techniques, to generate a thermo stable and active enzymes. PROSS algorithm (Goldenzweig, A. et al. Automated Structure- and Sequence-Based Design of Proteins for High Bacterial Expression and Stability. Mol. Cell 2016, 63 (2), 337-346) was utilized. Since WT ColG Xray structure lacks the Ca2+ ion, while a water molecule is located in its cavity, the water was replaced with a Ca2+ Ion.

The following modified collagenases were generated: Des1 (SEQ ID NO:3 or 13), Des4 (SEQ ID NO: 6 or 16) and Des6 (SEQ ID NO: 8 or 18).

Des1 includes 15 amino acid replacements/substitutions (approx. 2.2% of the protein). Most of the replacements were positioned on the surface of the protein. Surface exposed hydrophobic (or less hydrophilic) amino acids were mutated/substituted to more hydrophilic or even charged residues, raising solubility and thermal resistance, e.g. F295Y, A334D, S353H, T635N, Q669D, G670N, G672T, S701N and A709E. The latter can form a hydrogen bond with Y693 from an adjacent α helix. Moreover, the N203Y mutation can form pi-pi stacking with Tyr150 from a neighboring loop. Lastly, introducing prolines rigidify backbone stability, therefore A458P and D536P were introduced as well. (pssm=position-specific scoring matrix).

The mutations introduced in Des1 sequence are listed in Table 1:

TABLE 1

| Position | WT | Des1 | |
|---|---|---|---|
| 334 | A | D | 1st pssm surface polarity H bond |
| | | | internal bb |
| 458 | A | P | 1st pssm loop rigidity |
| 709 | A | E | 1st pssm with K. H bond with Y693 |
| 536 | D | P | 1st pssm.loop rigidity |
| 737 | D | K | 1st pssm |
| 710 | E | H | 1st pssm H bond with N419. |
| | | | (nitrogen)) H bond network H710- |
| | | | N419-E709(bb) |
| 295 | F | Y | 1st pssm.surface polarity |
| 670 | G | N | 1st pssm.surface polarity |
| 672 | G | T | 1st pssm.surface polarity H bond with |
| | | | bb of I673 |
| 183 | M | D | 1st pssm.surface polarity H bond with |
| | | | internal bb D183 and bb of G185 |
| 149 | N | Q | 1st pssm.surface flexibility and |
| | | | stability |
| 203 | N | Y | 3rd pssm pi stacking |
| 287 | N | Y | 1st pssm. H bond with Lys 291 also |
| | | | surface polarity |
| 669 | Q | D | 1st pssm surface polarity |
| 353 | S | H | 1st pssm.surface polarity |
| 701 | S | N | 1st pssm.surface polarity |
| 635 | T | N | 1st pssm.surface polarity |

### Example 4: Thermal Stability of a Modified Recombinant Collagenase Protein Vs a WT Recombinant Protein

A heat inactivation assay was performed to test for thermal stability of the recombinant proteins by preincubating the purified proteins, (recombinant collagenase (ColG) and the modified collagenase (Des1)), at temperatures ranging between 35 and 90° C. for 1 h. Residual activity was then measured by monitoring collagenase activity (as described in Tohar R. et. al., Int. J. Mol. Sci. 2021, 22, 8552).

The midpoint of temperature inactivation, the temperature at which 50% of the activity was retained (or lost) (T50), was determined by fitting a two-state model using GraphPad.

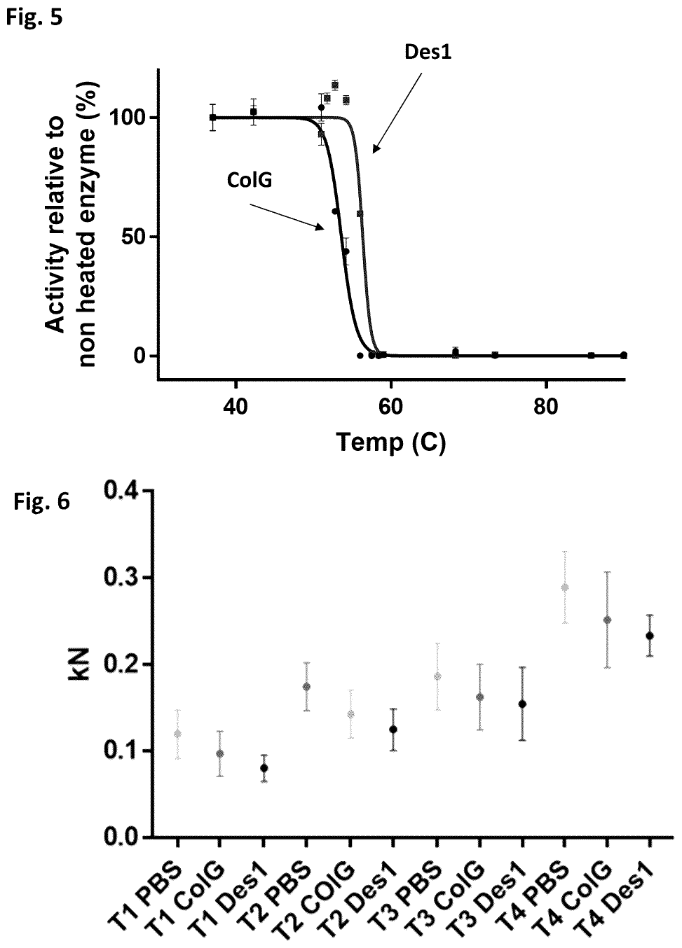

The results presented in FIG. 5, demonstrate that the T50 of the WT recombinant collagenase was about 53.6° C., whereas the T50 of the modified recombinant collagenase (Des1) was about 56.3° C.

The results clearly demonstrate that the modified collagenase was more thermally stable as compared to the recombinant collagenase, and hence exhibited improved physical and biochemical properties.

### Example 5—Qualitative and Quantitative Analysis of Tooth Extraction Forces after Administration of WT or Recombinant Collagenase

Experiments on Mandibles of six-month-old (90-100 kg) domestic swine were performed similarly to those detailed in the materials and method section and in Examples 2-3.

Recombinant collagenase (ColG) or modified recombinant collagenases (Des1, Des 4 or Des 6) or PBS were injected with the Wand Single Tooth Anesthesia System (Milestone Scientific, New Jersey, USA) (FIG. 1C). Standard cartridges containing the local anesthetic solution for dental injection were accurately emptied of their content and filled with either ColG or Des1 at a concentration of 4 μg/μl or PBS solution, followed by application to each treated root. Injection was performed using a 30G 2.54 cm length needle that was inserted into the PDL space and advanced apically until stopped by resistance of the alveolar bone proper. The injection was repeated at four sites around each root, on the buccal, lingual, mesial, and distal aspects. A total of 0.3 ml of 4 ug/ul ColG or PBS solution was injected.

A tooth extraction anchoring device was used, and measurement of the tooth extraction force was performed as detailed above.

The results presented in FIG. 6 demonstrate a comparison of the forces required to extract the different roots following the injection of PBS, the recombinant collagenase (ColG) or the modified recombinant collagenase (Des1). The results clearly and surprisingly show that the administration of the modified collagenase, Des1, dramatically reduces the extraction forces of each root as compared to a recombinant collagenase.

The results demonstrate that the modified recombinase exhibit improved biological activity as compared to the recombinase and exhibit enhanced effect when used in dental related procedures.

### Example 6—Evaluation of Cellular Viability

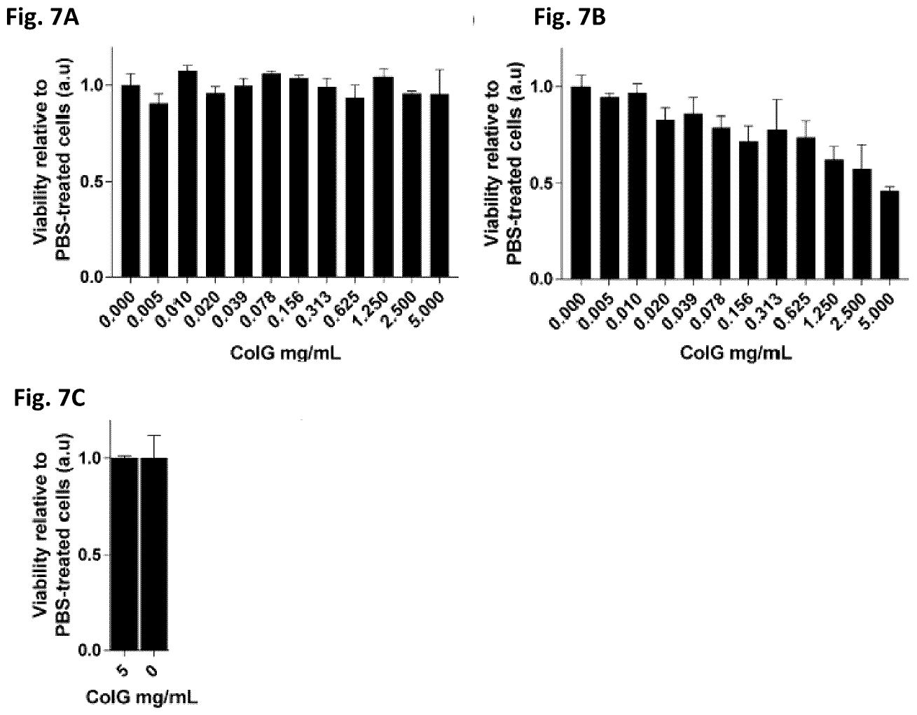

The effect of recombinant collagenase (ColG) on the viability of non-collagen-dependent and collagen-dependent cells was evaluated. FIG. 7A shows the viability of CHO cells treated with variable ColG concentrations relative to PBS-treated cells. The number of viable cells stayed at the same level and did not depend on ColG concentrations, thus demonstrating the safety of the ColG on non-collagen-dependent cells, such as CHO.

The effect of the enzyme on the principal cells inhabiting the gingiva and the mucosal tissue located in close proximity to the site of injection was of particular interest. Therefore, the viability of hGFs following treatment with ColG, with identical concentrations as applied to the CHO cells was evaluated (FIG. 7B). The degradation of collagen due to ColG activity hampered hGF vitality in a dose-dependent manner.

To determine if the effect of collagenase on hGFs was transient and that the growth rate would return to normal once ColG was depleted from the medium, 40,000 cells that had been treated with PBS or with the highest ColG concentration (5 mg/mL) were re-plated with a fresh medium that did not contain ColG. After 24 h, the viability of hGFs was measured again. FIG. 7C showed that the viability of ColG- and PBS-pre-treated cells was similar.

### Example 7—Cellular Toxicity

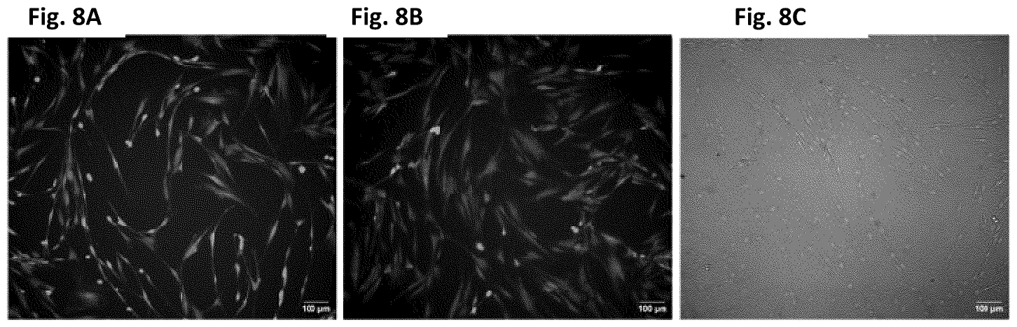

As a final validation that the enzyme was not toxic to the cells, the cellular death of hGFs were monitored. To this end, cells were seeded in a 6-well plate, 1,200,000 cells/well, and treated with ColG at a concentration of 5 mg/mL, PBS and 70% ethanol. Following overnight incubation, cellular images were recorded. FIG. 8 shows images of cells with the three treatments: hGFs treated with (FIG. 8A) 5 mg/mL ColG, (FIG. 8B) PBS or (FIG. 8C) 70% ethanol. Green cells are viable and red cells are dead. As demonstrated, collagenase did not result in cellular death on the hGFs.

Listed below are the numbering of the SEQ IDs of wild type, recombinant or modified recombinant collagenase forms, as disclosed herein:
Recombinant collagenase polypeptide (Amino Acids)—SEQ ID NO: 1
Recombinant collagenase nucleotide sequence (nucleic acids of coding sequence)—SEQ ID NO: 2
Modified recombinant collagenase polypeptide (Amino Acids) Des1—SEQ ID NO: 3
Modified Recombinant collagenase nucleotide sequence (Des1) SEQ ID NO: 4
Modified recombinant collagenase polypeptide (Amino Acids) Des4—SEQ ID NO: 5
Modified Recombinant collagenase nucleotide sequence (Des4) SEQ ID NO: 6
Modified recombinant collagenase polypeptide (Amino Acids) Des6—SEQ ID NO: 7
Modified Recombinant collagenase nucleotide sequence (Des6)—SEQ ID NO: 8
Clostridium WT collagenase nucleic acid sequence—SEQ ID NO: 9
Clostridium WT collagenase amino acid sequence—SEQ ID NO: 10
Artificial N-terminal region amino acid sequence—SEQ ID NO: 11:
M G S S H H H H H H S S G E N L Y F Q G G T M
(Underlined-His Tag, bold: TEV cleavage site).
Artificial N-terminal region nucleic acid sequence SEQ ID NO: 12:
atg ggc age age cat cat cat cat cat cac age age ggc gaa aac ctg tat ttt cag ggc ggt acc atg
Modified recombinant collagenase polypeptide (Amino Acids) Des1—SEQ ID NO: 13
Modified Recombinant collagenase nucleotide sequence (Des1) SEQ ID NO: 14
Modified recombinant collagenase polypeptide (Amino Acids) Des4—SEQ ID NO: 15
Modified Recombinant collagenase nucleotide sequence (Des4) SEQ ID NO: 16
Modified recombinant collagenase polypeptide (Amino Acids) Des6—SEQ ID NO: 17
Modified Recombinant collagenase nucleotide sequence (Des6) SEQ ID NO: 18

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1.-29. (canceled)

30. A modified recombinant collagenase polypeptide, said modified recombinant collagenase polypeptide comprising one or more amino acid replacements relative to a corresponding wild type collagenase amino acid sequence; and wherein the thermal stability of the modified recombinant collagenase polypeptide is higher than the thermal stability of a corresponding wild type (WT) polypeptide.

31. The modified recombinant collagenase polypeptide according to claim 30, comprising an N-terminal truncation, wherein the N-terminal truncation comprises a truncation of at least 15 amino acids relative to the corresponding wild type collagenase amino acid sequence.

32. The modified recombinant collagenase polypeptide according to claim 30, comprising an amino acid sequence as denoted by any one of SEQ ID NO: 3, 5, 7, 13, 15 or 17.

33. The modified recombinant collagenase polypeptide according to claim 30, comprising an amino acid sequence as denoted by SEQ ID NO: 3 or SEQ ID NO: 13.

34. The modified recombinant collagenase polypeptide according to claim 30, wherein the polypeptide further comprises a Tag sequence at the N-terminus and/or the C-terminus thereof.

35. The modified recombinant collagenase polypeptide according to claim 30, wherein the thermal stability of the modified recombinant collagenase polypeptide is higher than the thermal stability of a recombinant polypeptide comprising an N-terminal truncation.

36. A composition comprising the modified recombinant collagenase polypeptide according to claim 30.

37. A method of treating a dental related condition in a subject in need thereof, the method comprising administering to the subject in need thereof an effective amount of the modified recombinant collagenase polypeptide according to claim 30, or a composition comprising the same.

38. The method according to claim 37, wherein the dental related procedures is selected from tooth extraction, orthodontic procedure and implant removal.

39. The method according to claim 37, wherein the modified recombinant collagenase polypeptide or the composition is administered prior to, during or after the dental related procedure.

40. The method according to claim 37, wherein the modified recombinant collagenase polypeptide or the composition comprising the same is administered locally.

41. The method according to claim 40, wherein the administration is by local injection.

42. The method according to claim 37, wherein administration thereof reduces extraction force during tooth extraction, wherein the required extraction force is reduced compared to administration of a WT collagenase (having an amino acid sequence as denoted by SEQ ID NO: 10), a truncated recombinant collagenase (having an amino acid sequence as denoted by SEQ ID NO: 1), or to administration of a carrier.

43. The method according to claim 42, wherein the modified recombinant collagenase polypeptide or the composition comprising the same are administered locally to a periodontal ligament (PDL) region prior to the tooth extraction.

44. A nucleic acid molecule encoding the modified recombinant collagenase of claim 30.

45. The nucleic acid molecule according to claim 44, comprising a nucleotide sequence as denoted by any one of: SEQ ID NO: 4, SEQ ID NO: 6, SEQ ID NO: 8, SEQ ID NO: 14, SEQ ID NO: 16, or SEQ ID NO: 18.

46. A vector comprising the nucleic acid molecule of claim 44.

47. A host cell comprising the nucleic acid molecule according to claim 44.

48. A host cell transformed or transfected with the vector according to claim 46.

49. A host cell comprising the modified recombinant collagenase polypeptide according to claim 30.


SEQUENCE LISTING (18 sequences)

SEQ ID NO: 1 - protein, 1023 aa, synthetic construct
MGSSHHHHHHSSGENLYFQGGTMYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKLILGYLAKDKVDHRYSLKKTLNSGYDDSDWMFYNYGFAVAHYLYEKDMPTFIKMNKAILNTDVKSYDEIIKKLSDDANKNTEYQNHIQELVDKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 2 - DNA, 3072 bp, synthetic construct
  source 1..3072: mol_type = other DNA
atgggcagcagccatcatcatcatcatcacagcagcggcgaaaacctgtattttcagggcggtaccatgtatgattttgagtatttaaacggtttgagctatactgaacttacaaatttaattaaaaatataaagtggaatcaaattaatggtttatttaattatagtacaggttctcaaaagttctttggagataaaaatcgtgtacaagctataattaatgctttacaagaaagtggaagaacttacactgcaaatgatatgaagggtatagaaactttcactgaggttttaagagctggtttttatttagggtactataatgatggtttatcttatttaaatgatagaaacttccaagataaatgtatacctgcaatgattgcaattcaaaaaaatcctaactttaagctaggaactgcagttcaagatgaagttataacttctttaggaaaactaataggaaatgcttctgctaatgctgaagtagttaataattgtgtaccagttctaaaacaatttagagaaaacttaaatcaatatgctcctgattacgttaaaggaacagctgtaaatgaattaattaaaggtattgaattcgatttttctggtgctgcatatgaaaaagatgttaagacaatgccttggtatggaaaaattgatccatttataaatgaacttaaggccttaggtctatatggaaatataacaagtgcaactgagtgggcatctgatgttggaatatactatttaagtaaattcgggctttactcaactaaccgaaatgacatagtacagtcacttgaaaaggctgtagatatgtataagtatggtaaaatagcctttgtagcaatggagagaataacttgggattatgatgggattggttctaatggtaaaaaggtggatcacgataagttcttagatgatgctgaaaaacattatctgccaaagacatatacttttgataatggaacctttattataagagcaggggataaggtatccgaagaaaaaataaaaaggctatattgggcatcaagagaagtgaagtctcaattccatagagtagttggcaatgataaagctttagaggtgggaaatgccgatgatgttttaactatgaaaatatttaatagcccagaagaatataaatttaataccaatataaatggtgtaagtactgataatggtggtctatatatagaaccaagagggactttctacacttatgagagaacacctcaacaaagtatatttagtcttgaagaattgtttagacatgaatatactcactatttacaagcgagatatcttgtagatggtttatgggggcaaggtccattttatgaaaaaaatagattaacttggtttgatgaaggtacagctgaattctttgcaggatctacccgtacatctggtgttttaccaagaaaattaatattaggatatttggctaaggataaagtagatcatagatactcattaaagaagactcttaattcagggtatgatgacagtgattggatgttctataattatggatttgcagttgcacattacctatatgaaaaagatatgcctacatttattaagatgaataaagctatattgaatacagatgtgaaatcttatgatgaaataataaaaaaattaagtgatgatgcaaataaaaatacagaatatcaaaaccatattcaagagttagtagataaatatcaaggagcaggcatacctctagtatcagatgattacttaaaagatcatggatataagaaagcatctgaagtatattctgaaatttcaaaagctgcttctcttacaaacactagtgtaacagcagaaaaatctcaatactttaacacattcactttaagaggaacttatacaggtgaaacttctaaaggtgaatttaaagattgggatgaaatgagtaaaaaattagatggaactttggagtcccttgctaaaaattcttggagtggatacaaaactttaacagcatactttacgaattatagagttacaagcgataataaagttcaatatgatgtagttttccatggggttttaacagataatggggatattagtaacaataaggctccaatagcaaaggtaactggaccaagcactggtgctgtaggaagaaatattgaatttagtggaaaagatagtaaagatgaagatggtaaaatagtatcatatgattgggattttggcgatggtgcaactagtagaggcaaaaattcagtacatgcttacaaaaaagcaggaacatataatgttacattaaaagtaactgacgataagggtgcaacagctacagaaagctttactatagaaataaagaacgaagatacaacaacacctataactaaagaaatggaacctaatgatgatataaaagaggctaatggtccaatagttgaaggtgttactgtaaaaggtgatttaaatggttctgatgatgctgataccttctattttgatgtaaaagaagatggtgatgttacaattgaacttccttattcagggtcatctaatttcacatggttagtttataaagagggagacgatcaaaatcatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa

SEQ ID NO: 3 - protein, 1000 aa, synthetic construct
YDFEYLNGLSYTELTNLIKNIKWNQINGLFQYSTGSQKFFGDKNRVQAIINALQESGRTYTANDDKGIETFTEVLRAGFYLGYYYDGLSYLNDRNFQDKCIPAMIAIQKNPNFKLGTAVQDEVITSLGKLIGNASANAEVVNNCVPVLKQFRENLNQYAPDYVKGTAVYELIKGIEYDFSGAAYEKDVKTMPWYGKIDPFINELKALGLYGNITSDTEWASDVGIYYLSKFGLYHTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKPLEVGNADDVLTMKIFNSPEEYKFNTNINGVSTDNGGLYIEPRGTFYTYERTPQQSIFSLEELFRHEYTHYLQARYLVPGLWGQGPFYEKNRLTWFDEGTAEFFAGSTRTSGVLPRKLILGYLAKDKVDHRYSLKKTLNSGYDDSDWMFYNYGFAVAHYLYEKDMPTFIKMNKAILNNDVKSYDEIIKKLSDDANKNTEYQNHIQELVDKYDNATIPLVSDDYLKDHGYKKASEVYSEISKAANLTNTSVTEHKSQYFNTFTLRGTYTGETSKGEFKDWKEMSKKLDGTLESLAKNSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 4 - DNA, 3003 bp, synthetic construct
  source 1..3003: mol_type = other DNA
tacgactttgaatatctgaacggtctgagttatactgaactcaccaatcttattaaaaacatcaaatggaaccagattaatggcttgtttcagtattccaccggtagccagaagttttttggagataaaaatcgcgttcaggctatcattaatgcgctccaggaatccggtcgcacctacaccgccaatgacgataaagggatcgaaacattcactgaagtgctgcgcgcgggattttatttggggtactattatgatggcttaagctatcttaatgatcgcaattttcaggacaaatgcattccggcaatgattgctatccagaaaaatccgaattttaaattaggtactgctgtgcaggacgaagtgattaccagcctgggtaagttaatcggcaatgcttctgccaatgctgaagtagttaacaattgcgtgccggtcctgaaacagttccgtgagaatctgaatcagtatgcgccagactatgttaaaggcacagctgtttacgaattgattaaaggcatcgaatatgacttctccggtgcagcatacgagaaggatgttaaaaccatgccttggtacggaaagattgacccatttattaacgaattgaaagctctgggtctgtatggtaatatcacatccgacaccgaatgggcctcagacgttggcatttactatctgagcaaattcggtctctatcataccaatcgcaacgacatcgttcagagcctcgagaaggcggttgacatgtataaatacggtaagatcgcattcgtggcaatggaacggattacctgggactatgacggtatcggctcgaatggtaagaaggttgaccatgacaaatttctggatgacgccgaaaaacactacctgccgaaaacctatactttcgataatggcacttttattattcgtgcaggcgataaagtgtccgaagagaaaatcaaacgcttatattgggcgagccgggaagttaaatcacaatttcatcgtgttgtgggtaacgataaacctctggaagtgggtaatgccgacgacgtcctgacgatgaaaatctttaactccccagaagaatataaatttaacactaatatcaacggcgtttctactgacaatggtggtttgtatattgaaccccgtggcacattctatacctatgaacgcacccctcagcagtcaatcttctcgttagaggaacttttccgccacgaatacactcactatctccaggcacgttatttagtgccaggcctgtggggccaaggccctttctacgaaaagaaccgtctgacttggtttgatgagggtacggcggaattcttcgcggggagtacccgtactagtggtgtgcttccacgtaagttgatcctgggttatttggcgaaagataaggtagaccaccgttactccctgaagaaaacactgaactccgggtatgacgatagtgattggatgttttacaactacggcttcgcggttgcccattatctgtacgaaaaagatatgcccacgtttattaaaatgaacaaagccattctgaacaacgacgtgaaaagctatgatgaaattattaagaaactcagtgatgatgcaaataaaaacactgaataccagaaccacatccaggagctggtcgacaaatatgacaatgctacgattccactggtgagcgatgattacctgaaagaccatggctacaaaaaggcatccgaagtgtactcggagatttccaaagcggcgaacctgacgaatacatctgttaccgaacataagagccagtactttaacacctttaccctgcgtggaacttatacgggcgaaacgagcaaaggcgagtttaaagattggaaagaaatgtccaaaaaactggatggcacgttagagtccttggccaaaaacagctggtccggttataaaacgctgaccgcttattttactaattaccgggtaacatccgacaacaaggtgcagtacgacgtggtctttcacggcgtcctcacggataacggggatatttcgaacaataaagcgccgatcgcgaaagttaccgggccaagtacgggcgcggtgggacgtaacatcgaattttctgggaaagactctaaagatgaagatggtaaaattgttagttatgactgggatttcggggacggcgcgactagccgtggcaagaatagcgtacacgcctataaaaaagccggcacctacaacgtgacgctgaaagttacggacgacaagggcgccacagccacggaatcttttaccatcgaaattaaaaatgaagataccaccacgccgattacgaaagaaatggaacctaacgatgatattaaagaagccaacggacctatcgtggaaggtgtcaccgttaaaggcgatttaaacggaagcgatgatgctgatacattttattttgatgtcaaggaagatggggatgtaacaattgaacttccttattcagggtcatctaatttcacatggttagtttataaagagggagacgatcaaaatcatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa

SEQ ID NO: 5 - protein, 1000 aa, synthetic construct
YDFEYLNGLSYTELTNLIKNIKWNQINGLFQYSDGSQKFYGDKNRVQAIINALQESGRTYTANDDKGIETFTEVLRAGFYLGYYYDGLSYLNDRNFQDKCIPAMIAIQKNPNFKLGTAVQDEVIASLGKLIGNASANAEVVNNCVPVLKQFRENLNQYAPDYVKGTAVYELIKGIEYDFSGAAYEKDVKTMPWYGKIDPFINELKKLGLYGNITSDTEWASDVGIYYLSKFGLYHTNRNDIVQALEKAVDMYKYGKIAFVAMERIKWDYDGIGSNGKKVDHDKFLEDAEKHYLPKTYTFDNGTFIIRAGDKVSEEKIKRLYWASKEVKAQFHRVVGNDKPLEVGNADDVLTMKIYNSPEEYKFNTYINGVSTDNGGLYIEPRGTFYTYERTPQQSIFSLEELFRHEYTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEIIKKLSDDANKNKEYQNHIQELVDRYDNATIPLVSDDYLKDHGYKPASEVYSEISKAANLTNTSVTKHKSQFFNTFTLRGTYTGGTSKGEFKDWKEMSKKLDETLESLSKKSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 6 - DNA, 3003 bp, synthetic construct
  source 1..3003: mol_type = other DNA
tacgatttcgaatacctgaacggcctgtcttacacagaactgaccaatttaatcaaaaatattaaatggaatcagattaatggactgttccagtacagcgatggcagtcaaaaattttatggtgataaaaaccgtgtgcaggctatcatcaatgctttacaagagagtggccggacatacaccgcgaatgatgataaaggcattgagacctttaccgaagttttacgcgctgggttctatctcggttattactacgatgggttgagttacctgaacgatcgcaatttccaggataaatgtattccggccatgattgcgattcagaaaaatccgaactttaaactgggcaccgctgttcaggacgaagtaatcgcgagcctgggcaaactgatcggcaatgcgtcagccaacgcggaagtggtgaacaattgcgtcccggtgcttaaacagttccgtgaaaatctgaaccagtacgccccggactatgtgaagggcacagcggtgtatgaacttattaagggcattgaatatgacttctcgggtgccgcatacgaaaaagacgtgaaaacaatgccgtggtacggtaaaatcgatccgtttattaacgaactgaaaaagctgggcctgtacggcaatatcacaagcgatacggaatgggcgtctgacgtcggcatctactatctgtctaagtttggtctgtatcataccaatcgtaacgatattgtgcaggccctggagaaagctgtcgacatgtacaaatatggcaaaattgctttcgtggccatggagcgcatcaagtgggactacgacgggattggaagcaacggcaagaaggtagatcatgataagtttctcgaagatgccgaaaaacattaccttccgaaaacgtacacgtttgataacggaacgtttattattcgtgctggggataaagtaagtgaagaaaaaatcaaacgcttgtattgggcgagcaaagaggtcaaagcgcagttccaccgcgtggtaggcaacgataaaccactggaggtaggcaacgcagatgatgtccttacaatgaagatctacaactcgccggaggaatataaatttaatacttatattaacggtgtctccactgataatggcggtctctatatcgaaccgcgcggaaccttttatacatatgagcgcactccacagcaaagcatcttctccctggaagaactgtttcgtcatgaatatacccattaccttcaggcgcgttatttagtgccagggttatggggccaaggcccattttacgagaacaatcggctgacgtggttcgatgaaggtacagcagagttttttgcagggagtacgcgcacgtccggcgttttaccgcgtaaaactattctgggttacctggcgaaagacaaagttgatcatcgttattctctgaaaaaaactctgaactccggctatgacgattctgattggatgttttataactatggttttgccgtggcccactacctgtatgaaaaggatatgccgacctttatcaagatgcacaaagccattttaaacaatgacgttaaatcctacgacgaaatcatcaaaaaactgtcggatgatgcgaacaaaaacaaggaataccagaatcatatccaggaactggtagaccgttacgacaacgcaactatccccctcgtatctgacgattacctgaaagatcatggctacaaacccgcgtcggaagtgtacagcgaaatctcaaaagctgcaaacctgacgaatacgtcagtcactaagcacaaatcacaatttttcaacacgttcaccctgcgcggtacctatacgggcgggacgtctaaaggcgaatttaaagattggaaagagatgtcgaaaaaattagacgagaccctggagagtctgtcgaaaaagagctggtcgggatataaaaccttaactgcatatttcaccaactatcgtgttactagcgacaacaaagtgcaatatgacgtggtctttcacggtgtcctgactgataacggagacatcagtaataataaggcaccgattgctaaagttaccgggcctagcactggcgctgtcgggcgcaatattgaatttagcggtaaagatagcaaagacgaagatggcaagattgtgtcatacgattgggactttggtgatggtgccacgtcgcgtgggaagaactcggttcatgcgtataaaaaagcgggcacctacaacgttacccttaaagtgaccgatgacaaaggggcgaccgcgaccgaaagtttcaccatcgaaatcaagaacgaggatacgaccacgccgatcacgaaagagatggaaccgaacgatgatattaaggaagcgaatggcccgatcgtggaaggcgtaaccgtcaagggcgacctgaacggctccgatgatgctgataccttttactttgatgtcaaggaggatggcgatgtcacaattgaacttccttattcagggtcatctaatttcacatggttagtttataaagagggagacgatcaaaatcatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa

SEQ ID NO: 7 - protein, 1000 aa, synthetic construct
YDFEYLNGLSYDELTNLIKNIKWNQINGLFQYSDGSQKFYGDKNRVQAIINALEESGRTYTANDDKGIETFTEVLRAGFYLGYYYDGLSYLNDRNFQDKCIPAMIAIQKNPNFKLGTDVQDEVIAALGKLIGNASANAEVVNNCVPVLKQFRENLNQYAPDYSKGTAVYELIKGIEYDFSGAAYEKDPKTMPWYGKIDPFINELKKLGLYGNITSDTEWASNVGIYYLSKFGKYHSNRNDIVQALEKAVDMYKYGKIAFVAMERIKEDYDGIGSNGKKVDHDKFKEDAEKHYLPKTYTFDNGTFIIRAGDKVSEEKIKRLYWASKEVKAQFHRVVGNDKPLEVGNADDVLTMKIYNSPEEYKFNTYINGVSTDNGGIYIEPRGTFYTYERTPQQSIFSLEELFRHEFTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEYIKKLSDDANKNKEYQNHIQELVDRYDNATIPLVSDDYLKDHGYKPASEIYSEIAKAANLTNTSVTKHKSQFFNTFTLRGTYTGGTSKGEFQDWKEMNKKLDEILEQLSKKSWSGYKTLTAYFTNYRVTSDNQVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 8 - DNA, 3003 bp, synthetic construct
  source 1..3003: mol_type = other DNA
tatgatttcgaatacctgaatgggttgagttacgacgaactgacgaatctgatcaaaaacatcaaatggaatcagattaatggactgtttcagtactccgatggcagccagaaattttatggtgacaaaaatcgtgtgcaggcaatcattaacgcactcgaagaaagtggacgtacttatactgcgaacgacgataaaggcattgaaacctttaccgaagtgcttcgcgcgggtttctacctgggctactattacgatggcctgtcctatctgaacgaccgtaattttcaggacaagtgtattcctgccatgatcgcgattcagaagaacccgaactttaaactggggactgatgtgcaggacgaggtgatcgcggccctgggcaaattgatcggtaatgcgtcggcaaacgctgaagtcgtcaacaactgtgttccggtcctgaaacaatttcgcgaaaaccttaatcagtatgcgcctgattactccaaaggtactgccgtttacgagcttattaaaggcattgaatacgacttttcgggcgcagcctacgaaaaagacccgaaaacgatgccatggtatgggaaaattgatccgtttatcaacgaattgaaaaaactgggactttatggcaacattacgtcagataccgaatgggcgagcaacgttggtatctactacctgagcaaattcggtaaatatcatagcaatcgcaacgacatcgtccaggccctcgaaaaagccgttgatatgtacaaatacggtaaaatcgcgtttgtggccatggaacgcatcaaagaagactatgacggcatcggtagcaatgggaagaaagttgatcatgataagttcaaggaagacgctgaaaaacattatctcccaaaaacctacacctttgacaacgggaccttcattattcgggcaggcgataaagtgtcggaggaaaaaatcaaacgcctgtactgggcatcaaaagaggtgaaggcccaattccatcgtgtggtgggtaatgataaaccgcttgaagtggggaacgcggacgatgttctgacgatgaagatttataactcccccgaagaatacaagtttaacacatacattaacggggtcagtaccgataacgggggtatttacatcgaaccacgcggtacgttctacacgtacgaacgtacccctcagcagtctattttctcgctggaagaattgtttcgccatgagtttacccattatctgcaagcgcgttatttagttccaggtttatggggccaaggtccgttctatgagaacaatcgcttaacttggtttgacgaaggcacggcggaatttttcgccggatccacccggactagcggcgttcttccccgtaagaccatcctgggctacctcgctaaagataaagtggaccaccgttattccctgaagaaaactcttaactccggctacgatgactcggattggatgttttataactatggctttgccgttgcgcattatttatatgaaaaagacatgccgacgtttattaaaatgcacaaagccatcttaaataacgatgttaaatcctacgatgagtatattaaaaaattgtcagacgatgctaacaagaacaaagaataccagaaccacatccaggaacttgtggatcggtatgataacgccaccatcccgttagtgagcgatgattacctgaaagaccacggatacaaaccagcgtcagaaatttatagcgaaatcgctaaagccgcgaatctgacaaacacctctgtgactaaacataaatcacagttcttcaatacgttcactcttcgcggcacttatacggggggcacgagcaaaggagaattccaagattggaaagaaatgaacaaaaaactcgatgaaatcctggaacaactgtcgaaaaaatcatggagtggttataaaacgctgactgcctacttcaccaattaccgcgttacgagcgacaatcaggttcaatacgacgtagtcttccacggtgtccttactgataacggcgatatcagcaataacaaagccccaattgctaaagtgaccggcccgagtacgggcgcagtgggtcgcaacatcgaattctctggtaaagattcgaaagatgaagacggtaaaatcgtgtcgtatgattgggattttggggatggtgcgacatctcgcggtaaaaacagcgtgcacgcttataaaaaagcaggcacgtacaatgtgacgcttaaagtgaccgatgacaagggtgctaccgcgaccgagagcttcaccattgaaattaagaacgaagatacaaccaccccgattaccaaagagatggaaccgaatgacgatatcaaagaagcgaatggccctattgtcgaaggagtcactgtaaaaggcgatctgaacggtagcgacgatgcggataccttctattttgacgttaaagaagacggtgatgttacaattgaacttccttattcagggtcatctaatttcacatggttagtttataaagagggagacgatcaaaatcatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa

SEQ ID NO: 9 - DNA, 3354 bp, Clostridium histolyticum
  source 1..3354: mol_type = genomic DNA
atgaaaaaaaatatattaaaaatattaatggatagttatagtaaagaaagtaaaatacaaacagttagaagagttacaagtgttagtttattagcagtttatttaacaatgaatacaagtagtttagttttagcaaaacctatagaaaatacaaatgatacaagtataaaaaatgttgaaaaattaagaaatgcacctaatgaagaaaatagtaaaaaagttgaagatagtaaaaatgataaagttgaacatgttaaaaatatagaagaagcaaaagttgaacaagttgcacctgaagttaaaagtaaaagtacattaagaagtgcaagtatagcaaatacaaatagtgaaaaatatgattttgaatatttaaatggattaagttatacagaattaacaaatttaataaaaaatataaaatggaatcaaataaatggattatttaattatagtacaggaagtcaaaaattttttggagataaaaatagagttcaagcaataataaatgcattacaagaaagtggaagaacatatacagcaaatgatatgaaaggaatagaaacatttacagaagttttaagagcaggattttatttaggatattataatgatggattaagttatttaaatgatagaaattttcaagataaatgtatacctgcaatgatagcaatacaaaaaaatcctaattttaaattaggaacagcagttcaagatgaagttataacaagtttaggaaaattaataggaaatgcaagtgcaaatgcagaagttgttaataattgtgttcctgttttaaaacaatttagagaaaatttaaatcaatatgcacctgattatgttaaaggaacagcagttaatgaattaataaaaggaatagaatttgattttagtggagcagcatatgaaaaagatgttaaaacaatgccttggtatggaaaaatagatccttttataaatgaattaaaagcattaggattatatggaaatataacaagtgcaacagaatgggcaagtgatgttggaatatattatttaagtaaatttggattatatagtacaaatagaaatgatatagttcaaagtttagaaaaagcagttgatatgtataaatatggaaaaatagcatttgttgcaatggaaagaataacatgggattatgatggaataggaagtaatggaaaaaaagttgatcatgataaatttttagatgatgcagaaaaacattatttacctaaaacatatacatttgataatggaacatttataataagagcaggagataaagttagtgaagaaaaaataaaaagattatattgggcaagtagagaagttaaaagtcaatttcatagagttgttggaaatgataaagcattagaagttggaaatgcagatgatgttttaacaatgaaaatatttaatagtcctgaagaatataaatttaatacaaatataaatggagttagtacagataatggaggattatatatagaacctagaggaacattttatacatatgaaagaacacctcaacaaagtatatttagtttagaagaattatttagacatgaatatacacattatttacaagcaagatatttagttgatggattatggggacaaggacctttttatgaaaaaaatagattaacatggtttgatgaaggaacagcagaattttttgcaggaagtacaagaacaagtggagttttacctagaaaaagtatattaggatatttagcaaaagataaagttgatcatagatatagtttaaaaaaaacattaaatagtggatatgatgatagtgattggatgttttataattatggatttgcagttgcacattatttatatgaaaaagatatgcctacatttataaaaatgaataaagcaatattaaatacagatgttaaaagttatgatgaaataataaaaaaattaagtgatgatgcaaataaaaatacagaatatcaaaatcatatacaagaattagcagataaatatcaaggagcaggaatacctttagttagtgatgattatttaaaagatcatggatataaaaaagcaagtgaagtttatagtgaaataagtaaagcagcaagtttaacaaatacaagtgttacagcagaaaaaagtcaatattttaatacatttacattaagaggaacatatacaggagaaacaagtaaaggagaatttaaagattgggatgaaatgagtaaaaaattagatggaacattagaaagtttagcaaaaaatagttggagtggatataaaacattaacagcatattttacaaattatagagttacaagtgataataaagttcaatatgatgttgtttttcatggagttttaacagataatgcagatataagtaataataaagcacctatagcaaaagttacaggacctagtacaggagcagttggaagaaatatagaatttagtggaaaagatagtaaagatgaagatggaaaaatagttagttatgattgggattttggagatggagcaacaagtagaggaaaaaatagtgttcatgcatataaaaaagcaggaacatataatgttacattaaaagttacagatgataaaggagcaacagcaacagaaagttttacaatagaaataaaaaatgaagatacaacaacacctataacaaaagaaatggaacctaatgatgatataaaagaagcaaatggacctatagttgaaggagttacagttaaaggagatttaaatggaagtgatgatgcagatacattttattttgatgttaaagaagatggagatgttacaatagaattaccttatagtggaagtagtaattttacatggttagtttataaagaaggagatgatcaaaatcatatagcaagtggaatagataaaaataatagtaaagttggaacatttaaaagtacaaaaggaagacattatgtttttatatataaacatgatagtgcaagtaatataagttatagtttaaatataaaaggattaggaaatgaaaaattaaaagaaaaagaaaataatgatagtagtgataaagcaacagttatacctaattttaatacaacaatgcaaggaagtttattaggagatgatagtagagattattatagttttgaagttaaagaagaaggagaagttaatatagaattagataaaaaagatgaatttggagttacatggacattacatcctgaaagtaatataaatgatagaataacatatggacaagttgatggaaataaagttagtaataaagttaaattaagacctggaaaatattatttattagtttataaatatagtggaagtggaaattatgaattaagagttaataaa

SEQ ID NO: 10 - protein, 1118 aa, Clostridium histolyticum
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 11 - protein, 23 aa, synthetic construct
MGSSHHHHHHSSGENLYFQGGTM

SEQ ID NO: 12 - DNA, 69 bp, synthetic construct
  source 1..69: mol_type = other DNA
atgggcagcagccatcatcatcatcatcacagcagcggcgaaaacctgtattttcagggcggtaccatg

SEQ ID NO: 13 - protein, 1023 aa, synthetic construct
MGSSHHHHHHSSGENLYFQGGTMYDFEYLNGLSYTELTNLIKNIKWNQINGLFQYSTGSQKFFGDKNRVQAIINALQESGRTYTANDDKGIETFTEVLRAGFYLGYYYDGLSYLNDRNFQDKCIPAMIAIQKNPNFKLGTAVQDEVITSLGKLIGNASANAEVVNNCVPVLKQFRENLNQYAPDYVKGTAVYELIKGIEYDFSGAAYEKDVKTMPWYGKIDPFINELKALGLYGNITSDTEWASDVGIYYLSKFGLYHTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKPLEVGNADDVLTMKIFNSPEEYKFNTNINGVSTDNGGLYIEPRGTFYTYERTPQQSIFSLEELFRHEYTHYLQARYLVPGLWGQGPFYEKNRLTWFDEGTAEFFAGSTRTSGVLPRKLILGYLAKDKVDHRYSLKKTLNSGYDDSDWMFYNYGFAVAHYLYEKDMPTFIKMNKAILNNDVKSYDEIIKKLSDDANKNTEYQNHIQELVDKYDNATIPLVSDDYLKDHGYKKASEVYSEISKAANLTNTSVTEHKSQYFNTFTLRGTYTGETSKGEFKDWKEMSKKLDGTLESLAKNSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 14 - DNA, 3072 bp, synthetic construct
  source 1..3072: mol_type = other DNA
atgggcagcagccatcatcatcatcatcacagcagcggcgaaaacctgtattttcagggcggtaccatgtacgactttgaatatctgaacggtctgagttatactgaactcaccaatcttattaaaaacatcaaatggaaccagattaatggcttgtttcagtattccaccggtagccagaagttttttggagataaaaatcgcgttcaggctatcattaatgcgctccaggaatccggtcgcacctacaccgccaatgacgataaagggatcgaaacattcactgaagtgctgcgcgcgggattttatttggggtactattatgatggcttaagctatcttaatgatcgcaattttcaggacaaatgcattccggcaatgattgctatccagaaaaatccgaattttaaattaggtactgctgtgcaggacgaagtgattaccagcctgggtaagttaatcggcaatgcttctgccaatgctgaagtagttaacaattgcgtgccggtcctgaaacagttccgtgagaatctgaatcagtatgcgccagactatgttaaaggcacagctgtttacgaattgattaaaggcatcgaatatgacttctccggtgcagcatacgagaaggatgttaaaaccatgccttggtacggaaagattgacccatttattaacgaattgaaagctctgggtctgtatggtaatatcacatccgacaccgaatgggcctcagacgttggcatttactatctgagcaaattcggtctctatcataccaatcgcaacgacatcgttcagagcctcgagaaggcggttgacatgtataaatacggtaagatcgcattcgtggcaatggaacggattacctgggactatgacggtatcggctcgaatggtaagaaggttgaccatgacaaatttctggatgacgccgaaaaacactacctgccgaaaacctatactttcgataatggcacttttattattcgtgcaggcgataaagtgtccgaagagaaaatcaaacgcttatattgggcgagccgggaagttaaatcacaatttcatcgtgttgtgggtaacgataaacctctggaagtgggtaatgccgacgacgtcctgacgatgaaaatctttaactccccagaagaatataaatttaacactaatatcaacggcgtttctactgacaatggtggtttgtatattgaaccccgtggcacattctatacctatgaacgcacccctcagcagtcaatcttctcgttagaggaacttttccgccacgaatacactcactatctccaggcacgttatttagtgccaggcctgtggggccaaggccctttctacgaaaagaaccgtctgacttggtttgatgagggtacggcggaattcttcgcggggagtacccgtactagtggtgtgcttccacgtaagttgatcctgggttatttggcgaaagataaggtagaccaccgttactccctgaagaaaacactgaactccgggtatgacgatagtgattggatgttttacaactacggcttcgcggttgcccattatctgtacgaaaaagatatgcccacgtttattaaaatgaacaaagccattctgaacaacgacgtgaaaagctatgatgaaattattaagaaactcagtgatgatgcaaataaaaacactgaataccagaaccacatccaggagctggtcgacaaatatgacaatgctacgattccactggtgagcgatgattacctgaaagaccatggctacaaaaaggcatccgaagtgtactcggagatttccaaagcggcgaacctgacgaatacatctgttaccgaacataagagccagtactttaacacctttaccctgcgtggaacttatacgggcgaaacgagcaaaggcgagtttaaagattggaaagaaatgtccaaaaaactggatggcacgttagagtccttggccaaaaacagctggtccggttataaaacgctgaccgcttattttactaattaccgggtaacatccgacaacaaggtgcagtacgacgtggtctttcacggcgtcctcacggataacggggatatttcgaacaataaagcgccgatcgcgaaagttaccgggccaagtacgggcgcggtgggacgtaacatcgaattttctgggaaagactctaaagatgaagatggtaaaattgttagttatgactgggatttcggggacggcgcgactagccgtggcaagaatagcgtacacgcctataaaaaagccggcacctacaacgtgacgctgaaagttacggacgacaagggcgccacagccacggaatcttttaccatcgaaattaaaaatgaagataccaccacgccgattacgaaagaaatggaacctaacgatgatattaaagaagccaacggacctatcgtggaaggtgtcaccgttaaaggcgatttaaacggaagcgatgatgctgatacattttattttgatgtcaaggaagatggggatgtaacaattgaacttccttattcagggtcatctaatttcacatggttagtttataaagagggagacgatcaaaatcatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa

SEQ ID NO: 15 - protein, 1023 aa, synthetic construct
MGSSHHHHHHSSGENLYFQGGTMYDFEYLNGLSYTELTNLIKNIKWNQINGLFQYSDGSQKFYGDKNRVQAIINALQESGRTYTANDDKGIETFTEVLRAGFYLGYYYDGLSYLNDRNFQDKCIPAMIAIQKNPNFKLGTAVQDEVIASLGKLIGNASANAEVVNNCVPVLKQFRENLNQYAPDYVKGTAVYELIKGIEYDFSGAAYEKDVKTMPWYGKIDPFINELKKLGLYGNITSDTEWASDVGIYYLSKFGLYHTNRNDIVQALEKAVDMYKYGKIAFVAMERIKWDYDGIGSNGKKVDHDKFLEDAEKHYLPKTYTFDNGTFIIRAGDKVSEEKIKRLYWASKEVKAQFHRVVGNDKPLEVGNADDVLTMKIYNSPEEYKFNTYINGVSTDNGGLYIEPRGTFYTYERTPQQSIFSLEELFRHEYTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEIIKKLSDDANKNKEYQNHIQELVDRYDNATIPLVSDDYLKDHGYKPASEVYSEISKAANLTNTSVTKHKSQFFNTFTLRGTYTGGTSKGEFKDWKEMSKKLDETLESLSKKSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 16 - DNA, 3072 bp, synthetic construct
  source 1..3072: mol_type = other DNA
atgggcagcagccatcatcatcatcatcacagcagcggcgaaaacctgtattttcagggcggtaccatgtacgatttcgaatacctgaacggcctgtcttacacagaactgaccaatttaatcaaaaatattaaatggaatcagattaatggactgttccagtacagcgatggcagtcaaaaattttatggtgataaaaaccgtgtgcaggctatcatcaatgctttacaagagagtggccggacatacaccgcgaatgatgataaaggcattgagacctttaccgaagttttacgcgctgggttctatctcggttattactacgatgggttgagttacctgaacgatcgcaatttccaggataaatgtattccggccatgattgcgattcagaaaaatccgaactttaaactgggcaccgctgttcaggacgaagtaatcgcgagcctgggcaaactgatcggcaatgcgtcagccaacgcggaagtggtgaacaattgcgtcccggtgcttaaacagttccgtgaaaatctgaaccagtacgccccggactatgtgaagggcacagcggtgtatgaacttattaagggcattgaatatgacttctcgggtgccgcatacgaaaaagacgtgaaaacaatgccgtggtacggtaaaatcgatccgtttattaacgaactgaaaaagctgggcctgtacggcaatatcacaagcgatacggaatgggcgtctgacgtcggcatctactatctgtctaagtttggtctgtatcataccaatcgtaacgatattgtgcaggccctggagaaagctgtcgacatgtacaaatatggcaaaattgctttcgtggccatggagcgcatcaagtgggactacgacgggattggaagcaacggcaagaaggtagatcatgataagtttctcgaagatgccgaaaaacattaccttccgaaaacgtacacgtttgataacggaacgtttattattcgtgctggggataaagtaagtgaagaaaaaatcaaacgcttgtattgggcgagcaaagaggtcaaagcgcagttccaccgcgtggtaggcaacgataaaccactggaggtaggcaacgcagatgatgtccttacaatgaagatctacaactcgccggaggaatataaatttaatacttatattaacggtgtctccactgataatggcggtctctatatcgaaccgcgcggaaccttttatacatatgagcgcactccacagcaaagcatcttctccctggaagaactgtttcgtcatgaatatacccattaccttcaggcgcgttatttagtgccagggttatggggccaaggcccattttacgagaacaatcggctgacgtggttcgatgaaggtacagcagagttttttgcagggagtacgcgcacgtccggcgttttaccgcgtaaaactattctgggttacctggcgaaagacaaagttgatcatcgttattctctgaaaaaaactctgaactccggctatgacgattctgattggatgttttataactatggttttgccgtggcccactacctgtatgaaaaggatatgccgacctttatcaagatgcacaaagccattttaaacaatgacgttaaatcctacgacgaaatcatcaaaaaactgtcggatgatgcgaacaaaaacaaggaataccagaatcatatccaggaactggtagaccgttacgacaacgcaactatccccctcgtatctgacgattacctgaaagatcatggctacaaacccgcgtcggaagtgtacagcgaaatctcaaaagctgcaaacctgacgaatacgtcagtcactaagcacaaatcacaatttttcaacacgttcaccctgcgcggtacctatacgggcgggacgtctaaaggcgaatttaaagattggaaagagatgtcgaaaaaattagacgagaccctggagagtctgtcgaaaaagagctggtcgggatataaaaccttaactgcatatttcaccaactatcgtgttactagcgacaacaaagtgcaatatgacgtggtctttcacggtgtcctgactgataacggagacatcagtaataataaggcaccgattgctaaagttaccgggcctagcactggcgctgtcgggcgcaatattgaatttagcggtaaagatagcaaagacgaagatggcaagattgtgtcatacgattgggactttggtgatggtgccacgtcgcgtgggaagaactcggttcatgcgtataaaaaagcgggcacctacaacgttacccttaaagtgaccgatgacaaaggggcgaccgcgaccgaaagtttcaccatcgaaatcaagaacgaggatacgaccacgccgatcacgaaagagatggaaccgaacgatgatattaaggaagcgaatggcccgatcgtggaaggcgtaaccgtcaagggcgacctgaacggctccgatgatgctgataccttttactttgatgtcaaggaggatggcgatgtcacaattgaacttccttattcagggtcatctaatttcacatggttagtttataaagagggagacgatcaaaatcatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa

SEQ ID NO: 17 - protein, 1023 aa, synthetic construct
MGSSHHHHHHSSGENLYFQGGTMYDFEYLNGLSYDELTNLIKNIKWNQINGLFQYSDGSQKFYGDKNRVQAIINALEESGRTYTANDDKGIETFTEVLRAGFYLGYYYDGLSYLNDRNFQDKCIPAMIAIQKNPNFKLGTDVQDEVIAALGKLIGNASANAEVVNNCVPVLKQFRENLNQYAPDYSKGTAVYELIKGIEYDFSGAAYEKDPKTMPWYGKIDPFINELKKLGLYGNITSDTEWASNVGIYYLSKFGKYHSNRNDIVQALEKAVDMYKYGKIAFVAMERIKEDYDGIGSNGKKVDHDKFKEDAEKHYLPKTYTFDNGTFIIRAGDKVSEEKIKRLYWASKEVKAQFHRVVGNDKPLEVGNADDVLTMKIYNSPEEYKFNTYINGVSTDNGGIYIEPRGTFYTYERTPQQSIFSLEELFRHEFTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEYIKKLSDDANKNKEYQNHIQELVDRYDNATIPLVSDDYLKDHGYKPASEIYSEIAKAANLTNTSVTKHKSQFFNTFTLRGTYTGGTSKGEFQDWKEMNKKLDEILEQLSKKSWSGYKTLTAYFTNYRVTSDNQVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 18 - DNA, 3072 bp, synthetic construct
  source 1..3072: mol_type = other DNA
atgggcagcagccatcatcatcatcatcacagcagcggcgaaaacctgtattttcagggcggtaccatgtatgatttcgaatacctgaatgggttgagttacgacgaactgacgaatctgatcaaaaacatcaaatggaatcagattaatggactgtttcagtactccgatggcagccagaaattttatggtgacaaaaatcgtgtgcaggcaatcattaacgcactcgaagaaagtggacgtacttatactgcgaacgacgataaaggcattgaaacctttaccgaagtgcttcgcgcgggtttctacctgggctactattacgatggcctgtcctatctgaacgaccgtaattttcaggacaagtgtattcctgccatgatcgcgattcagaagaacccgaactttaaactggggactgatgtgcaggacgaggtgatcgcggccctgggcaaattgatcggtaatgcgtcggcaaacgctgaagtcgtcaacaactgtgttccggtcctgaaacaatttcgcgaaaaccttaatcagtatgcgcctgattactccaaaggtactgccgtttacgagcttattaaaggcattgaatacgacttttcgggcgcagcctacgaaaaagacccgaaaacgatgccatggtatgggaaaattgatccgtttatcaacgaattgaaaaaactgggactttatggcaacattacgtcagataccgaatgggcgagcaacgttggtatctactacctgagcaaattcggtaaatatcatagcaatcgcaacgacatcgtccaggccctcgaaaaagccgttgatatgtacaaatacggtaaaatcgcgtttgtggccatggaacgcatcaaagaagactatgacggcatcggtagcaatgggaagaaagttgatcatgataagttcaaggaagacgctgaaaaacattatctcccaaaaacctacacctttgacaacgggaccttcattattcgggcaggcgataaagtgtcggaggaaaaaatcaaacgcctgtactgggcatcaaaagaggtgaaggcccaattccatcgtgtggtgggtaatgataaaccgcttgaagtggggaacgcggacgatgttctgacgatgaagatttataactcccccgaagaatacaagtttaacacatacattaacggggtcagtaccgataacgggggtatttacatcgaaccacgcggtacgttctacacgtacgaacgtacccctcagcagtctattttctcgctggaagaattgtttcgccatgagtttacccattatctgcaagcgcgttatttagttccaggtttatggggccaaggtccgttctatgagaacaatcgcttaacttggtttgacgaaggcacggcggaatttttcgccggatccacccggactagcggcgttcttccccgtaagaccatcctgggctacctcgctaaagataaagtggaccaccgttattccctgaagaaaactcttaactccggctacgatgactcggattggatgttttataactatggctttgccgttgcgcattatttatatgaaaaagacatgccgacgtttattaaaatgcacaaagccatcttaaataacgatgttaaatcctacgatgagtatattaaaaaattgtcagacgatgctaacaagaacaaagaataccagaaccacatccaggaacttgtggatcggtatgataacgccaccatcccgttagtgagcgatgattacctgaaagaccacggatacaaaccagcgtcagaaatttatagcgaaatcgctaaagccgcgaatctgacaaacacctctgtgactaaacataaatcacagttcttcaatacgttcactcttcgcggcacttatacggggggcacgagcaaaggagaattccaagattggaaagaaatgaacaaaaaactcgatgaaatcctggaacaactgtcgaaaaaatcatggagtggttataaaacgctgactgcctacttcaccaattaccgcgttacgagcgacaatcaggttcaatacgacgtagtcttccacggtgtccttactgataacggcgatatcagcaataacaaagccccaattgctaaagtgaccggcccgagtacgggcgcagtgggtcgcaacatcgaattctctggtaaagattcgaaagatgaagacggtaaaatcgtgtcgtatgattgggattttggggatggtgcgacatctcgcggtaaaaacagcgtgcacgcttataaaaaagcaggcacgtacaatgtgacgcttaaagtgaccgatgacaagggtgctaccgcgaccgagagcttcaccattgaaattaagaacgaagatacaaccaccccgattaccaaagagatggaaccgaatgacgatatcaaagaagcgaatggccctattgtcgaaggagtcactgtaaaaggcgatctgaacggtagcgacgatgcggataccttctattttgacgttaaagaagacggtgatgttacaattgaacttccttattcagggtcatctaatttcacatggttagtttataaagagggagacgatcaaaatcatattgcaagtggtatagataagaataactcaaaagttggaacatttaaagctacaaaaggaagacattatgtgtttatatataaacacgattctgcttcaaatatatcctattctttaaacataaaaggattaggtaacgagaaattgaaggaaaaagaaaataatgattcttctgataaagctacagttataccaaatttcaataccactatgcaaggttcacttttaggtgatgattcaagagattattattcttttgaggttaaggaagaaggcgaagttaatatagaactagataaaaaggatgaatttggtgtaacatggacactacatccagagtcaaatattaatgacagaataacttacggacaagttgatggtaataaggtatctaataaagttaaattaagaccaggaaaatattatctacttgtttataaatactcaggatcaggaaactatgagttaagggtaaataaataa